(19) United States
(12) Patent Application Publication
(10) Pub. No.: US 2013/0243874 A1
(43) Pub. Date: Sep. 19, 2013

(54) NANOPARTICLES COATED WITH AMPHIPHILIC BLOCK COPOLYMERS

(71) Applicants: IMRA of America, Inc., Ann Arbor, MI (US); The Regents of the University of Michigan, Ann Arbor, MI (US)

(72) Inventors: Duxin Sun, Ann Arbor, MI (US); Hongwei Chen, Ann Arbor, MI (US); Masayuki Ito, Cupertino, CA (US); Wei Qian, Ann Arbor, MI (US); Yong Che, Ann Arbor, MI (US)

(73) Assignees: IMRA of America, Inc., Ann Arbor, MI (US); The Regents of the University of Michigan, Ann Arbor, MI (US)

(21) Appl. No.: 13/783,563

(22) Filed: Mar. 4, 2013

Related U.S. Application Data
(60) Provisional application No. 61/607,108, filed on Mar. 6, 2012.

(51) Int. Cl. A61K 47/02; A61K 9/14; G01N 21/47

(52) U.S. Cl. CPC A61K 47/02; G01N 21/47; A61K 9/143
USPC 424/497; 436/501; 252/408.1; 424/649

(57) ABSTRACT

The present provides amphiphilic block copolymer coated surfaces (e.g., nanoparticles, medical devices, etc.) and methods of preparing such surfaces. In certain embodiments, the present invention provides amphiphilic block copolymer coated single dispersed nanoparticles, which are stable in buffer (e.g., PBS) and have neutral but functionable surfaces, and methods of preparing the same.

The present application claims priority to U.S. Provisional application serial number 61/607,108, filed Mar. 6, 2012, which is herein incorporated by reference in its entirety.

This invention was made with government support under contracts nos. CA120023 and CA143474 awarded by the National Institutes of Health. The government has certain rights in the invention.

### TECHNICAL FIELD

The present invention relates to amphiphilic block copolymer coated surfaces (e.g., nanoparticles) and methods of preparing such surfaces. In certain embodiments, the present invention provides amphiphilic block copolymer coated single dispersed nanoparticles (e.g., gold nanoparticles), which are stable in buffer and have neutral but functionable surfaces, and methods of preparing the same.

### BACKGROUND

Gold nanoparticles have attracted substantial interest from scientists for over a century because of their unique physical, chemical, and surface properties, such as: (i) size- and shape-dependent strong optical extinction and scattering which is tunable from ultraviolate (UV) wavelengths all the way to near infrared (NIR) wavelengths; (ii) large surface areas for conjugation to functional ligands; and (iii) little or no long-term toxicity or other adverse effects in vivo allowing their high acceptance level in living systems. Gold nanoparticles are now being widely investigated for their potential use in various applications as imaging contrast agents (Nat. Biotechnol. 2008, 26, 83 and Nano Lett. 2005, 5, 829), therapeutic agents (Nano Lett. 2007, 7, 1929 and Sci. Trans'. Med. 2010, 2), biological sensors (Chem. Soc. Rev. 2008, 37, 2028), and cell-targeting vectors (Nano Lett. 2007, 7, 247). For both in vitro and in vivo applications, gold nanoparticles are usually coated with a polymeric layer to protect them from aggregation in physiological conditions or to further conjugation with targeting ligands to generate targeting nanoparticles (Langmuir 2007, 23, 5352, Langmuir 2006, 22, 11022, Nano Lett. 2005, 5, 473, Chem. Commun. 2007, 4580, Langmuir 2007, 23, 7491, Small 2011, 7, 2412, and Nanoscale Res. Lett. 2011, 6). Traditionally, these nanoparticles are coated with polymer containing reactive functional groups, such as —COOH and —NH₂, which are ready for the conjugation of targeting ligands (Nat. Biotechnol. 2008, 26, 83, J. Phys. Chem. C 2008, 112, 8127, J. Am. Chem. Soc. 2007, 129, 2871, and ACS Nano 2010, 4, 5887). However, nanoparticles with highly charged surfaces promote their binding to biomolecules in the biological systems through ionic interactions, causing nanoparticles to aggregate in biological environments (J. Mater. Chem. 2010, 20, 255), and thus exhibit strong non-specific binding to various cells and tissues that is undesirable in many in vitro and in vivo applications (J. Am. Chem. Soc. 2001, 123, 4103 and J. Am. Chem. Soc. 2007, 129, 3333).

To reduce non-specific binding, nanoparticles with a neutralized coating are favorable. A common approach is to conjugate multiple poly(ethylene oxide) (PEO) molecules with no polar groups onto the nanoparticle surface (Pharma. Res. 2007, 24, 1405, Biomaterials 2009, 30, 2340, and Adv. Mater. 2007, 19, 3163). However, most of them are not functional for further ligand conjugation. In order to functionalize the nanoparticles, carboxyl or amine modified PEO has to be used, which simultaneously increases the surface charge of PEO stabilized nanoparticles (ACS Nano 2010, 4, 5887). Although PEGylated gold nanoparticles prevent aggregation, the poor stability of gold nanoparticles, which occurs in the subsequently repeated conjugation process for functionalization of surface and in vivo application, is still one of the major challenges for its successful applications. Furthermore, PEGlyated gold nanoparticles are not suitable for encapsulate other therapeutic drug molecules without conjugation.

Currently, the overwhelming majority of gold nanoparticles are prepared by using the standard wet chemical sodium citrate reduction of tetrachloroaurate (HAuCl₄) methodology. This method results in the synthesis of spherical gold nanoparticles with diameters ranging from 5 to 200 nanometers (nm) which are capped or covered with negatively charged citrate ions. The citrate ion capping prevents the nanoparticles from aggregating by providing electrostatic repulsion. Other wet chemical methods for formation of gold nanoparticles include the Brust method, the Perrault method and the Martin method. The Brust method relies on reaction of chlorauric acid with tetraoctylammonium bromide in toluene and sodium borohydride. The Perrault method uses hydroquinone to reduce the HAuCl₄ in a solution containing gold nanoparticle seeds. The Martin method uses reduction of HAuCl₄ in water by NaBH₄ wherein the stabilizing agents HCl and NaOH are present in a precise ratio. All of the wet chemical methods rely on first converting gold (Au) with strong acid into the atomic formula HAuCl₄ and then using this atomic form to build up the nanoparticles in a bottom-up type of process. All of the methods require the presence of stabilizing agents to prevent the gold nanoparticles from aggregating and precipitating out of solution.

### SUMMARY OF THE INVENTION

The present disclosure provides amphiphilic block copolymer coated surfaces (e.g., nanoparticles, medical devices, etc.) and methods of preparing such surfaces. In certain embodiments, the present invention provides amphiphilic block copolymer coated single dispersed gold nanoparticles, which are stable in phosphate buffered saline (PBS) buffer and stable single dispersed gold nanoparticles with neutral but functionable surfaces, and methods of preparing the same.

In some embodiments, the present invention provides methods of producing stable amphiphilic block copolymer coated (e.g., single dispersed) gold nanoparticles comprising: a) preparing a stable colloidal suspension of gold nanoparticles in a organic solvent by a top-down nanofabrication method using bulk gold as a source material and preparing a solution of amphiphilic block copolymers in the organic solvent (e.g., the amphiphilic block copolymer contains at least one functional group having an affinity for surface of the gold nanoparticles in its hydrophobic part); b) mixing the solution of amphiphilic block copolymer with the colloidal suspension of gold nanoparticles (e.g., at room temperature for at least 8 hours), then treating the mixture at elevated temperature (e.g., for at least 2 hours), and then cooling the resultant mixture (e.g., to room temperature slowly). In certain embodiments, the treatment at elevated temperature enhancing the binding of the functional group in the amphiphilic block copolymer to the surface of the gold nanoparticle and enabling encapsulation of a single the gold nanoparticle in a shell formed by the amphiphilic block copolymers after transferring the resultant mixture into deionized water. In particular embodiments, the method further comprising: c) transferring the resultant mixture into aqueous solution by adding the resultant mixture dropwise to deionized water and then removing amphiphilic block copolymer coated single dispersed gold nanoparticles from the colloidal suspension. In particular embodiments, then the method comprises resuspending them in deionized water.

In some embodiments, the hydrophilic polymer block of the amphiphilic block copolymer comprise a plurality of polymers selected from but not limited to poly(2-(methacryloyloxy)ethyl phosphorylcholine), poly(2-(dimethylamino)ethyl methacrylate), poly(acrylic acid), poly(ethylene oxide), and poly(ethylene glycol). In certain embodiments, the hydrophobic polymer block of the amphiphilic block copolymers comprise a plurality of polymers selected from but not limited to poly(methyl methacrylate), polystyrene, poly(pyridyldisulfide ethylmethacrylate), poly(N-isopropylacrylamide), and poly(methacrylic acid). In other embodiments, the amphiphilic block copolymers comprise hydrophilic polymer block having degree of polymerization in the range from 1 unit to 250 units (e.g., 1 . . . 25 . . . 50 . . . 75 . . . 100 . . . 150 . . . 200 . . . 250). In further embodiments, the amphiphilic block copolymers comprise hydrophobic polymer block having degree of polymerization in the range from 1 unit to 100 units or 1 to 250 units.

In some embodiments, the stable amphiphilic block copolymer coated single dispersed gold nanoparticles have an absorbance intensity and wavelength caused by localized surface plasmon resonance of the amphiphilic block copolymer coated single dispersed gold nanoparticles in phosphate buffered saline (PBS) buffer upon storage for 72 hours that does not vary by more than plus or minus 10% (e.g., 1% . . . 4% . . . 8% . . . 10%) and 4 nanometers (e.g., 1, 2, 3, or 4 nanometers), respectively of the values as measured immediately after preparation of the amphiphilic block copolymer coated single dispersed gold nanoparticles in phosphate buffered saline (PBS) buffer. In certain embodiments, the stable colloidal suspension of gold nanoparticles in a organic solvent has an absorbance intensity and wavelength caused by localized surface plasmon resonance of a bare colloidal gold preparation upon storage for 72 hours that does not vary by more than plus or minus 10% and 4 nanometers, respectively of the values as measured after allowing as synthesis bare colloidal gold preparation to age for 1 week. In further embodiments, the organic solvents are selected from the group consisting of: methanol, ethanol, acetone, and dimethylformamide.

In some embodiments, the top-down nanofabrication methods comprise applying a physical energy source to a source of bulk gold in a organic solvent. In particular embodiments, the physical energy source comprising at least one of mechanical energy, heat energy, electric field arc discharge energy, magnetic field energy, ion beam energy, electron beam energy, or laser beam energy. In other embodiments, the top-down nanofabrication methods comprise a two-step process comprising first fabricating a gold nanoparticle array on a substrate by using photo, electron beam, focused ion beam, or nanosphere lithography and secondly removing the gold nanoparticle arrays from the substrate into a organic solvent. In further embodiments, the top-down nanofabrication methods comprise applying laser ablation to the source of bulk gold in a organic solvent. In other embodiments, the colloidal suspension of gold nanoparticles in a organic solvent comprises a population of gold nanoparticles wherein the gold nanoparticles have at least one dimension in the range of from 1 to 200 nanometers or from 1 to 400 nanometers. In

In some embodiments, the colloidal suspension of gold nanoparticles in a organic solvent comprises a population of gold nanoparticles wherein the shape of the gold nanoparticles comprises at least one of a sphere, a rod, a prism, a disk, a cube, a core-shell structure, a cage, a frame, or a mixture thereof. In other embodiments, the functional group having an affinity for surface of the gold nanoparticles comprises a thiol group, an amine group, a phosphine group, a disulfide group or a mixture thereof. In other embodiments, the treatment at elevated temperature comprises heating the mixture of the amphiphilic block copolymer and the colloidal suspension of gold nanoparticles to a temperature above about 60 degrees.

In some embodiments, the present invention provides amphiphilic block copolymer coated (e.g., single dispersed) gold nanoparticles (e.g., which are stable in phosphate buffered saline (PBS) buffer) comprising: a population of single gold nanoparticles encapsulated in a shell formed by the amphiphilic block copolymers, the amphiphilic block copolymers contains at least one functional group having an affinity for surface of the gold nanoparticles in its hydrophobic part. In other embodiments, the stable in phosphate buffered saline (PBS) buffer means that the absorbance intensity and wavelength caused by localized surface plasmon resonance of the amphiphilic block copolymer coated single dispersed gold nanoparticles in phosphate buffered saline (PBS) buffer upon storage for 72 hours does not vary by more than plus or minus 10% (e.g., 1% . . . 5% . . . 10%) and 4 nanometers, respectively of the values as measured immediately after preparation of the amphiphilic block copolymer coated single dispersed gold nanoparticles in phosphate buffered saline (PBS) buffer. In certain embodiments, the functional group having an affinity for surface of the gold nanoparticles comprises a thiol group, an amine group, a phosphine group, a disulfide group or a mixture thereof.

In some embodiments, the amphiphilic block copolymers are bound onto the surface of the gold nanoparticles by at least one of a thiol group, an amine group, a phosphine group, a disulfide group or a mixture thereof in hydrophobic parts of the amphiphilic block copolymer. In additional embodiments, the amphiphilic block copolymer comprises hydrophilic polymer block having a degree of polymerization in the range from 1 unit to 100 units or from 1 to 200 units. In certain embodiments, the amphiphilic block copolymer comprises hydrophobic polymer block having degree of polymerization in the range from 1 unit to 100 units. In further embodiments, the hydrophilic polymer block of the amphiphilic block copolymer comprise a plurality of polymers selected from the group consisting of: poly(2-(methacryloyloxy)ethyl phosphorylcholine), poly(2-(dimethylamino)ethyl methacrylate), poly(acrylic acid), poly(ethylene oxide), and poly(ethylene glycol). In other embodiments, the hydrophobic polymer block of the amphiphilic block copolymers comprise a plurality of polymers selected from the group consisting of: poly(methyl methacrylate), polystyrene, poly(pyridyldisulfide ethylmethacrylate), poly(N-isopropylacrylamide), and poly(methacrylic acid). In additional embodiments, the gold nanoparticles are prepared by a top-down nanofabrication method using bulk gold immersed in a organic solvent as a source material.

In some embodiments, the top-down nanofabrication method comprises applying a physical energy source to a source of bulk gold in a organic solvent, the physical energy source comprising at least one of mechanical energy, heat energy, electric field arc discharge energy, magnetic field energy, ion beam energy, electron beam energy, or laser beam energy. In further embodiments, the top-down nanofabrication methods comprise a two-step process comprising first fabricating a gold nanoparticle array on a substrate by using photo, electron beam, focused ion beam, or nanosphere lithography and secondly removing the gold nanoparticle arrays from the substrate into a organic solvent. In other embodiments, the top-down nanofabrication method comprises applying laser ablation to the source of bulk gold in a organic solvent. In other embodiments, the organic solvents comprise a plurality of solvents selected from the group consisting of: methanol, ethanol, acetone, and dimethylformamide.

In certain embodiments, the gold nanoparticles have at least one dimension in the range of from 1 to 200 nanometers or from 1 to 400 nanometers. In some embodiments, the shape of the nanoparticles comprises at least one of a sphere, a rod, a prism, a disk, a cube, a core-shell structure, a cage, a frame, or a mixture thereof. In other embodiments, the amphiphilic block copolymer coated single dispersed gold nanoparticles are a powder.

In certain embodiments, the present invention provide composition comprising, consisting of, or consisting essentially of: amphiphilic block copolymer poly(ethylene oxide)-block-poly(pyridyldisulfide ethylmethacrylate) (PEO-b-PPDSM).

In particular embodiments, the present invention provides methods for the preparation of amphiphilic block copolymer coated single dispersed gold nanoparticles. In certain embodiments, the produced amphiphilic block copolymer coated single dispersed gold nanoparticles have a size in at least one dimension of from 1 to 200 nanometers are stable in phosphate buffered saline (PBS) buffer for use in biological, medical, and other applications.

In some embodiments, the present invention provides a thiol-reactive amphiphilic block copolymer poly(ethylene oxide)-block-poly(pyridyldisulfide ethylmethacrylate) (PEO-b-PPDSM) coated surfaces and nanoparticles (e.g., single dispersed gold nanoparticles that have neutral but functionable surfaces and are stable in phosphate buffered saline (PBS) buffer). This poly(ethylene oxide)-block-poly(pyridyldisulfide ethylmethacrylate) (PEO-b-PPDSM) copolymer contains multiple disulfide bonds on PPDSM block which could form multiple Au—S interactions with metal nanoparticle (e.g., laser-ablated gold nanoparticles) to generate single dispersed nanoparticles with uniform size and high stability.

In other embodiments, the present invention provides surface functionalization of amphiphilic block copolymer poly(ethylene oxide)-block-poly(pyridyldisulfide ethylmethacrylate) (PEO-b-PPDSM) coated surfaces and nanoparticle (e.g., single dispersed gold nanoparticles) and to ability of copolymer coated gold nanoparticles to encapsulate hydrophobic therapeutic drugs.

In some embodiments, the present invention provides methods of producing stable amphiphilic block copolymer coated single dispersed nanoparticles comprising: a) mixing a solution of amphiphilic block copolymer with a colloidal suspension of nanoparticles (e.g., nanoparticles comprising gold, iron, nickel, cobalt; magnetic nanoparticles; or quantum dots) to generate a mixture, wherein the amphiphilic block copolymer comprises at least one functional group having an affinity for the nanoparticles; b) treating the mixture at a temperature of above about 60 degrees Celsius (e.g., 70 . . . 80 . . . 90 . . . 100 . . . 110 . . . 120 . . . 130 . . . 140 . . . 150 ... 160 . . . 170 . . . 180 . . . 190 or more) to generate a treated mixture; and c) adding at least a portion of the treated mixture to water (e.g., deionized water) such that a solution is generated that comprises amphiphilic block copolymer single dispersed nanoparticles.

In certain embodiments, the nanoparticles comprise gold nanoparticles. In other embodiments, the methods further comprise d) removing the amphiphilic block copolymer coated single dispersed nanoparticles from the solution and mixing with deionized water (e.g., placing the coated nanoparticles in a container of fresh deionized water). In certain embodiments, the treated mixture is added dropwise (or a similar slow introduction fashion) to the deionized water. In other embodiments, the deionized water is in motion (e.g., circular motion or other agitation) when the treated mixture is added thereto. In some embodiments, the temperature is above 100 degrees Celsius. In further embodiments, the temperature is about 60-160 degrees Celsius. In further embodiments the mixing in step a) is conducted at about room temperature.

In particular embodiments, the treated mixture is cooled to about room temperature after step b) but prior to step c). In other embodiments, the amphiphilic block copolymer comprises a polymer selected from the group consisting of: poly(2-(methacryloyloxy)ethyl phosphorylcholine), poly(2-(dimethylamino)ethyl methacrylate), poly(acrylic acid), poly(ethylene oxide), poly(ethylene glycol),poly(methyl methacrylate), polystyrene, poly(pyridyldisulfide ethylmethacrylate), poly(N-isopropylacrylamide), and poly(methacrylic acid). In other embodiments, the amphiphilic block copolymers comprise hydrophilic or hydrophobic polymer block having degree of polymerization in the range from 1 unit to 100 units (e.g., 1 . . . 25 . . . 50 . . . 75 . . . 95). In further embodiments, the methods further comprise, prior to step a), preparing the colloidal suspension of nanoparticles by a top-down nanofabrication method using bulk metal as a source material. In other embodiments, the top-down nanofabrication method comprises applying a physical energy source to the bulk metal, the physical energy source comprising at least one of mechanical energy, heat energy, electric field arc discharge energy, magnetic field energy, ion beam energy, electron beam energy, or laser beam energy.

In some embodiments, the colloidal suspension of nanoparticles comprises a population of nanoparticles wherein the nanoparticles have at least one dimension in the range of from 1 to 200 nanometers. In further embodiments, the functional group comprises a thiol group, an amine group, a phosphine group, a disulfide group or a mixture thereof.

In some embodiments, the present invention provides compositions comprising at least a portion of the amphiphilic block copolymer single dispersed nanoparticles prepared by the methods described herein.

In other embodiments, the present invention provides amphiphilic block copolymer coated single dispersed nanoparticles which are stable in buffer solution comprising: a population of single nanoparticles encapsulated in a shell formed by the amphiphilic block copolymers, the amphiphilic block copolymers contains at least one functional group having an affinity for the surface of the nanoparticles in its hydrophobic part and wherein the amphiphilic block copolymers coated nanoparticles have electrically neutralized surfaces and provide capability for further functionalization via thiol-disulfide exchange reactions.

In some embodiments, the functional group comprises a thiol group, an amine group, a phosphine group, a disulfide group or a mixture thereof. In certain embodiments, the amphiphilic block copolymer comprises hydrophobic or hydrophilic polymer block having degree of polymerization in the range from 1 unit to 100 units. In further embodiments, the hydrophilic or hydrophobic polymer block of the amphiphilic block copolymer comprise a plurality of polymers selected from the group consisting of: poly(2-(methacryloyloxy)ethyl phosphorylcholine), poly(2-(dimethylamino)ethyl methacrylate), poly(acrylic acid), poly(ethylene oxide), poly(ethylene glycol), poly(methyl methacrylate), polystyrene, poly(pyridyldisulfide ethylmethacrylate), poly(N-isopropylacrylamide), and poly(methacrylic acid).

In some embodiments, the nanoparticles have at least one dimension in the range of from 1 to 200 nanometers. In other embodiments, the amphiphilic block copolymer coated single dispersed nanoparticles are in powder form. In further embodiments, the nanoparticles comprise gold, quantum dots, iron, cobalt, or nickel.

### BRIEF DESCRIPTION OF THE DRAWINGS

FIG. 1. Schematic illustration of an exemplary laser-based ablation system for the top-down production of gold nanoparticles in a organic solvent.

FIG. 2. (a) Schematic illustration of polymerization of thiol-reactive block copolymer PEO-b-PPDSM using PEO macro-RAFT agent. (b) ¹H NMR spectrum of PEO-b-PPDSM in DMSO-d₆ (400 MHz). (c) Evolution of number-average molar mass (Mₙ) and polydispersity indexes (PDI) obtained by GPC for PEO macro-RAFT agent and the corresponding chain extended copolymer PEO-b-PPDSM. (d) Schematic representation of the preparation of PEO-b-PPDSM encapsulated gold nanoparticles.

FIG. 3. The absorption spectra of PEO-b-PPDSM coated gold nanoparticles before centrifugation (a) and after centrifugation (b). (c) The absorption spectra of supernatant after first time centrifugation. (d) Recovery of gold nanoparticles after centrifugation for three times.

FIG. 4. TEM images and gold nanoparticles size distributions before (a, b) and after heat treatment at 130° C. (degree) in DMF (c, d).

FIG. 5. The absorption spectra of the mixture solutions of (a) polymers and gold nanoparticles and (b) polymers only in DMF (with 10 times further dilution for absorption spectra) at different time points after heat treatment at 130° C. The data reveals that in addition to the consistent increase of optical density around 535 nm from gold plasma resonance with increasing time (a), another peak at ˜374 nm is also shown up (both a and b), revealing the release of pyridine-2-thione after heat treatment with reducing pyridyldisulfide bond. Based on the extinction coefficient of pyridine-2-thione in DMF, i.e. ε₃₇₄ₙₘ=5440 M⁻¹cm⁻¹, it is estimated that on average 0.8% of all the pyridyldisulfide bonds on polymer chains were reduced after heat treatment for 2 h.

FIG. 6. (a) TEM images of gold nanoparticles coated with copolymer PEO-b-PPDSM in water without negative staining under lower magnification with scale bar at 100 nm. (b) The particle size distribution of gold nanoparticles. (c) TEM image of negative staining nanoparticles under higher magnification with scale bar at 40 nm. (d) Hydrodynamic size distribution of polymeric micelles and polymer coated gold nanoparticles.

FIG. 7. Optical spectra of free doxorubicin (Dox) with different concentrations (a) and (b) the corresponding calibration curve. (c) Optical spectra of composite nanoparticles co-encapsulated with AuNP and 10% or 20% loading of Dox (neutral) in PBS and (d) their hydrodynamic size distribution. To load Dox, 1 mL or 2 mL of Dox solution (5.0 mg/mL in DMSO treated with TEA, 2 molar eq. to Dox·HCl) was added to the mixture (2 mL) of 50 mg of polymer and 20 nmol of gold nanoparticles after cooling to room temperature and then followed by transferring the organic solution to PBS (10 times volume to the organic solution) with pH 7.4 and dialysis against 2 L of PBS overnight.

FIG. 8. (a) Zeta potential of gold nanoparticles coated with PEO-b-PPDSM at different pH. (b) The absorption spectra of FITC-SH treated gold nanoparticles after washing with a 30K Nanosep filter until there is no detectable dye in the filtrated solution. The signal from FITC shows up compared to the spectrum of gold nanoparticles only, revealing the successful modification by disulfide linkage.

FIG. 9. UV-vis absorption spectra of amphiphilic block copolymer poly(ethylene oxide)-block-poly(pyridyldisulfide ethylmethacrylate) (PEO-b-PPDSM) coated gold nanoparticles show long term stability in phosphate buffered saline (PBS) buffer.

FIG. 10. Normalized optical density (OD) of gold nanoparticles coated with PEO-b-PPDSM or citrate after repeated centrifugation.

FIG. 11. The UV-vis spectrum of FITC only treated gold nanoparticles after washing with a 30K Nanosep filter for five times.

FIG. 12. Subtracted UV-vis spectrum of FITC-SH treated gold nanoparticles from gold nanoparticles only from FIG. 8b.

FIG. 13. Calibration curves of FTIC (a, b) and gold nanoparticles coated with PEO-b-PPDSM (c, d).

### DETAILED DESCRIPTION

The present provides amphiphilic block copolymer coated surfaces (e.g., nanoparticles, medical devices, etc.) and methods of preparing such surfaces. In certain embodiments, the present invention provides amphiphilic block copolymer coated single dispersed gold nanoparticles, which are stable in phosphate buffered saline (PBS) buffer and stable single dispersed gold nanoparticles with neutral but functionable surfaces, and methods of preparing the same.

Gold nanocolloids have attracted strong interest from scientists for over a century and are now being heavily investigated for their potential use in a wide variety of medical and biological applications. For example, potential uses include surface-enhanced spectroscopy, biological labeling and detection, gene-regulation, and diagnostic or therapeutic agents for treatment of cancer in humans. Their versatility in a broad range of applications stems from their unique physical, chemical, and surface properties, such as: (i) size- and shape-dependent strong optical extinction and scattering at visible and near infrared (NIR) wavelengths due to a localized surface plasmon resonance of their free electrons upon excitation by an electromagnetic field; (ii) large surface areas for conjugation to functional ligands; and (iii) little or no long-term toxicity or other adverse effects in vivo allowing their high acceptance level in living systems.

These new physical, chemical, and surface properties, which are not available from either atomic or bulk counterparts, explain why gold nanocolloids have not been simply chosen as alternatives to molecule-based systems but as novel structures which provide substantive advantages in biological and medical applications.

The prerequisite for most of intended biological and medical applications of gold nanoparticles is the further surface modification of the as-synthesized gold nanoparticles via conjugation of functional ligand molecules to the surface of the gold nanoparticles. The surface functionalization of gold nanoparticles for any biological or medical applications is crucial for at least two reasons. First is control over the interaction of the nanoparticles with their environment, which is naturally taking place at the nanoparticle surface. Appropriate surface functionalization is a key step to providing stability, solubility, and retention of physical and chemical properties of the nanoparticles in the physiological conditions. Second, the ligand molecules provide additional and new properties or functionality to those found inherently in the core gold nanoparticle. These conjugated gold nanoparticles bring together the unique properties and functionality of both the core material and the ligand shell for achieving the goals of highly specific targeting of gold nanoparticles to the sites of interest, ultra-sensitive sensing, and effective therapy.

Nowadays, the major strategy for surface modification of gold nanoparticles include coating gold nanoparticles with polymer containing reactive functional groups, such as —COOH and —NH₂, which are ready for the conjugation of targeting ligands. However, nanoparticles with highly charged surfaces promote their binding to biomolecules in the biological systems through ionic interactions, causing nanoparticles to aggregate in biological environments and thus exhibit strong non-specific binding to various cells and tissues that is undesirable in many in vitro and in vivo applications.

In certain embodiments, the present invention provides thiol-reactive amphiphilic block copolymer poly(ethylene oxide)-block-poly(pyridyldisulfide ethylmethacrylate) (PEO-b-PPDSM) coated nanoparticles (e.g., gold nanoparticles) with neutral but functional surfaces. In some embodiments, these nanoparticles are single dispersed with uniform particle size, are highly stable under physiological condition, have neutral but functionalizable surface, and have the ability to encapsulate therapeutic drugs.

As discussed above, the overwhelming majority of gold nanoparticles are prepared by the standard sodium citrate reduction reaction. This method allows for the synthesis of spherical gold nanoparticles with diameters ranging from about 5 to 200 nanometers (nm) which are capped with negatively charged citrate ions. The capping controls the growth of the nanoparticles in terms of rate, final size, geometric shape and stabilizes the nanoparticles against aggregation by electrostatic repulsion.

In contrast to the prior process of bottom-up fabrication using wet chemical processes, gold nanoparticles used in the present invention may be produced by a top-down nanofabrication approach. In certain embodiments, the top-down fabrication methods of the present invention start with a bulk material in a liquid and then break the bulk material into nanoparticles in the liquid by applying physical energy to the material. The physical energy can be mechanical energy, heat energy, electric field arc discharge energy, magnetic field energy, ion beam energy, electron beam energy, or laser beam energy including laser ablation of the bulk material. In some embodiments, the present process produces a pure, bare colloidal gold nanoparticle that is stable in the ablation liquid and avoids the wet chemical issues of residual chemical precursors, stabilizing agents and reducing agents. In certain embodiments, the ablation liquids comprise a plurality of solvents selected from but not limited to deionized water, methanol, ethanol, acetone, and dimethylformamide.

In certain embodiments, the nanocolloids (e.g., gold nanocolloids) produced by a top-down nanofabrication approach described in the present invention allows for production of stable nanocolloids with only partial surface modification to be fabricated. Also, the surface coverage amount of functional ligands on the surfaces of the fabricated gold nanoparticle conjugates can be tuned to be any percent value between 0 and 100%. In certain embodiments, the nanoparticles are gold particles produced by top-down nanofabrication approach which produces gold nanoparticle that are stable in the liquid they are created in with no need for stabilizing agents.

The present invention is not limited by the top-down nanofabrication techniques that are employed. In general, these techniques, require that the generation of the nanoparticles from the bulk material occur in the presence of the suspension medium. In one embodiment, the process comprises a one step process wherein the application of the physical energy source, such as mechanical energy, heat energy, electric field arc discharge energy, magnetic field energy, ion beam energy, electron beam energy, or laser energy to the bulk gold occur in the suspension medium. The bulk source is placed in the suspension medium and the physical energy is applied thus generating nanoparticles that are immediately suspended in the suspension medium as they are formed. In another embodiment the present invention is a two-step process including the steps of: 1) fabricating gold nanoparticle arrays on a substrate by using photo, electron beam, focused ion beam, nanoimprint, or nanosphere lithography as known in the art; and 2) removing the gold nanoparticle arrays from the substrate into the suspension liquid using one of the physical energy methods. Tabor, C., Qian, W., and El-Sayed, M. A., Journal of Physical Chemistry C, Vol 111 (2007), 8934-8941; Haes, A. J.; Zhao, J.; Zou, S. L.; Own, C. S.; Marks, L. D.; Schatz, G. C.; Van Duyne, R. P. Journal Of Physical Chemistry B, Vol 109 (2005), 11158. In both methods the colloidal gold is formed in situ by generating the nanoparticles in the suspension medium using one of the physical energy methods.

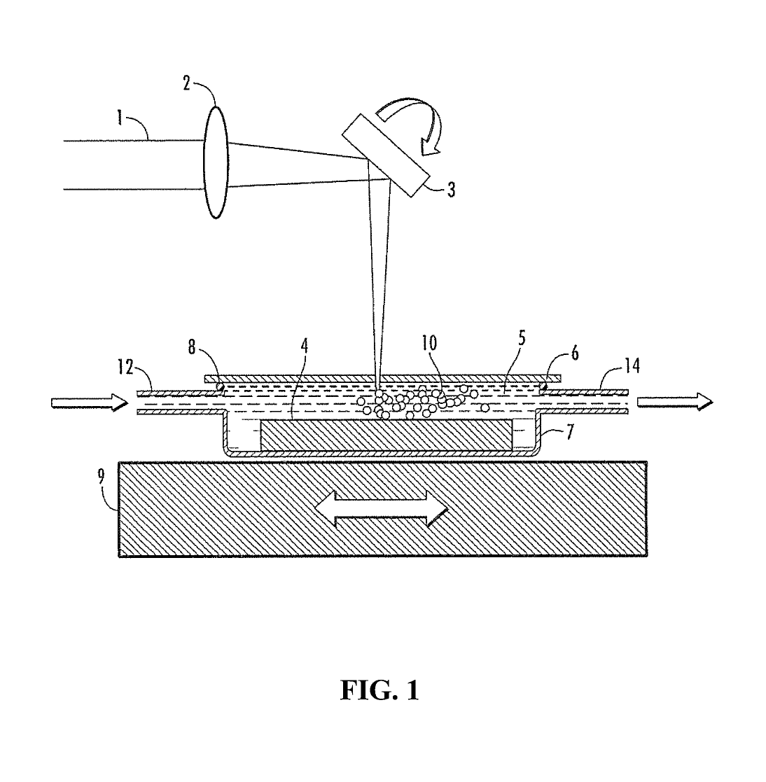

In work conducted during the development of embodiments of the present invention, colloidal suspension of gold nanoparticles were produced by pulsed laser ablation of a bulk gold target in acetone as the suspension medium. After a couple of days aging, the top clear red solution was transferred and mixed with dimethylformamide (DMF). Acetone was evaporated under reduced pressure to form a concentrate gold solution in DMF. FIG. 1 schematically illustrates a laser-based system for producing colloidal suspensions of nanoparticles of complex compounds such as gold in a organic liquid using pulsed laser ablation in accordance with the present invention. In one embodiment a laser beam 1 is generated from an ultrafast pulsed laser source, not shown, and focused by a lens 2. The source of the laser beam 1 can be a pulsed laser or any other laser source providing suitable pulse duration, repetition rate, and/or power level as discussed below. The focused laser beam 1 then passes from the lens 2 to a guide mechanism 3 for directing the laser beam 1. Alternatively, the lens 2 can be placed between the guide mechanism 3 and a target 4 of the bulk material. The guide mechanism 3 can be any of those known in the art including piezo-mirrors, acousto-optic deflectors, rotating polygons, a vibration mirror, or prisms. Preferably the guide mechanism 3 is a vibration mirror 3 to enable controlled and rapid movement of the laser beam 1. The guide mechanism 3 directs the laser beam 1 to a target 4. In one embodiment, the target 4 is a bulk gold target. The target 4 is submerged a distance, from several millimeters to preferably less than 1 centimeter, below the surface of a suspension organic liquid 5. The target 4 is positioned in a container 7 additionally but not necessarily having a removable glass window 6 on its top. Optionally, an O-ring type seal 8 is placed between the glass window 6 and the top of the container 7 to prevent the liquid 5 from leaking out of the container 7. Additionally but not necessarily, the container 7 includes an inlet 12 and an outlet 14 so the liquid 5 can be passed over the target 4 and thus be re-circulated. The container 7 is optionally placed on a motion stage 9 that can produce translational motion of the container 7 with the target 4 and the liquid 5. Flow of the liquid 5 is used to carry the nanoparticles 10 generated from the target 4 out of the container 7 to be collected as a colloidal suspension. The flow of organic liquid 5 over the target 4 also cools the laser focal volume. The organic liquid 5 can be any liquid that is largely transparent to the wavelength of the laser beam 1, and that serves as a colloidal suspension medium for the target material 4. In one embodiment, the liquid 5 is acetone. The system thus allows for generation of colloidal gold nanoparticles in situ in a suspension organic liquid so that a colloidal gold suspension is formed. The formed gold nanoparticles are immediately stably suspended in the organic liquid and thus no dispersants, stabilizer agents, surfactants or other materials are required to maintain the colloidal suspension in a stable state.

The following laser parameters were used to fabricate gold nanocolloids by pulsed laser ablation of a bulk gold target in acetone: pulse energy of 10 uJ (micro Joules), pulse repetition rate of 100 kHz, pulse duration of 700 femtoseconds, and a laser spot size on the ablation target of about 50 um (microns). For the preparation of Au nanocolloids, a 16 mm (millimeter) long, 8 mm wide, and 0.5 mm thick rectangular target of Au from Alfa Aesar was used. For convenience, the Au target materials can be attached to a bigger piece of a bulk material such as a glass slide, another metal substrate, or a Si substrate.

More generally, the laser ablation parameters may be as follows: a pulse duration in a range from about 10 femtoseconds to about 500 picoseconds, preferably from about 100 femtoseconds to about 30 picoseconds; the pulse energy in the range from about 1 μJ to about 100 μJ; the pulse repetition rate in the range from about 10 kHz to about 10 MHz; and the laser spot size may be less than about 100 μm. The target material has a size in at least one dimension that is greater than a spot size of a laser spot at a surface of the target material.

In certain embodiments, stable colloidal suspensions of bare gold nanoparticles can be created by a top-down fabrication method in situ in a organic solvent in the absence of stabilizing agents. Colloidal gold nanoparticles exhibit an absorbance peak in the wavelength range of 518 to 530 nanometers (nm). The term “stable” as applied to a colloidal gold preparation prepared according to the present invention refers to stability of the absorbance intensity caused by localized surface plasmon resonance of a bare colloidal gold preparation at 518 to 530 nm, more specifically at 520 nm upon storage. Generally, if a colloidal gold preparation becomes unstable the gold nanoparticles begin to aggregate and precipitate out of the suspension over time, thus leading to a decrease in the absorbance at 518-530 nm. In addition, “stable” means that there is a minimal red shift or change in localized surface plasmon resonance of 4 nanometers or less over storage time. In some embodiments, stable colloidal suspension of gold nanoparticles in a organic solvent prepared means that the absorbance intensity and wavelength caused by localized surface plasmon resonance of a bare colloidal gold preparation upon storage for 72 hours does not vary by more than plus or minus 10% and 4 nanometers, respectively of the values as measured after allowing as synthesis bare colloidal gold preparation to age for several days (typically about 1 week). The term “bare” as applied to the colloidal gold nanoparticles prepared according to the present invention means that the nanoparticles are pure gold with no surface modification or treatment other than creation as described in the liquid. The bare gold nanoparticles are also not in the presence of any stabilizing agents, they are simply in the preparation liquid which does not contain any nanoparticle stabilizers.

In the data described in this Examples below, amphiphilic block copolymers poly(ethylene oxide)-block-poly(pyridyldisulfide ethylmethacrylate) (PEO-b-PPDSM) contains pyridyldisulfide functional groups, were used, these were chosen for illustration purposes only. The invention is not limited to use amphiphilic block copolymers containing pyridyldisulfide functional groups for encapsulation of gold nanoparticles to form copolymer coated gold nanoparticles. Because the invention produces bare stable colloidal gold nanoparticles in organic solvent, any amphiphilic polymers having a functional group in their hydrophobic parts that can bind to Au particle surfaces can be used such as the suggested thiol groups, amine groups, or phosphine groups. In addition, the degree of polymerization of both hydrophilic and hydrophobic polymer block of amphiphilic block copolymer prefers to be in the range, for example, from 1 unit to 100 units (or more).

The coating of gold nanopartilcles described herein are not limited to application to only spherical colloidal Au nanoparticles having a diameter of from 1 to 200 nanometers. This method should also work for colloidal Au nanoparticles with other shapes and configurations, including rods, prisms, disks, cubes, core-shell structures, cages, and frames (e.g., wherein they have at least one dimension in the range of from 1 to 200 nm). In addition, the method of surface modification described in this invention should also work for nanostructures which have outer surfaces that are only partially covered with gold.

### EXAMPLES

### Example 1

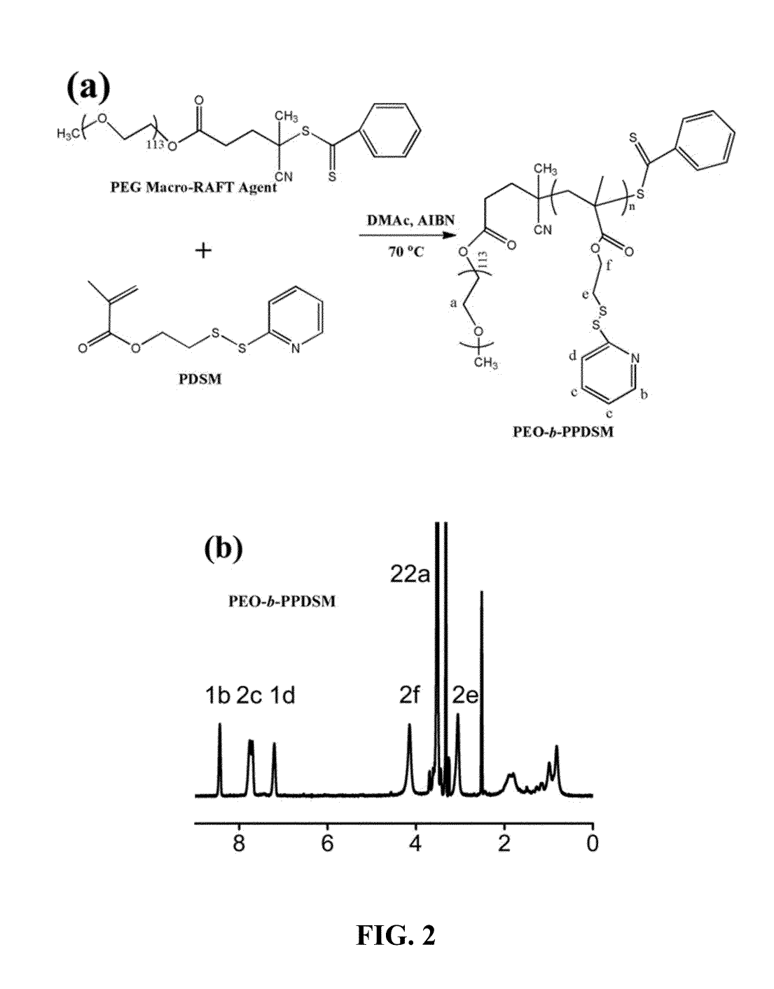

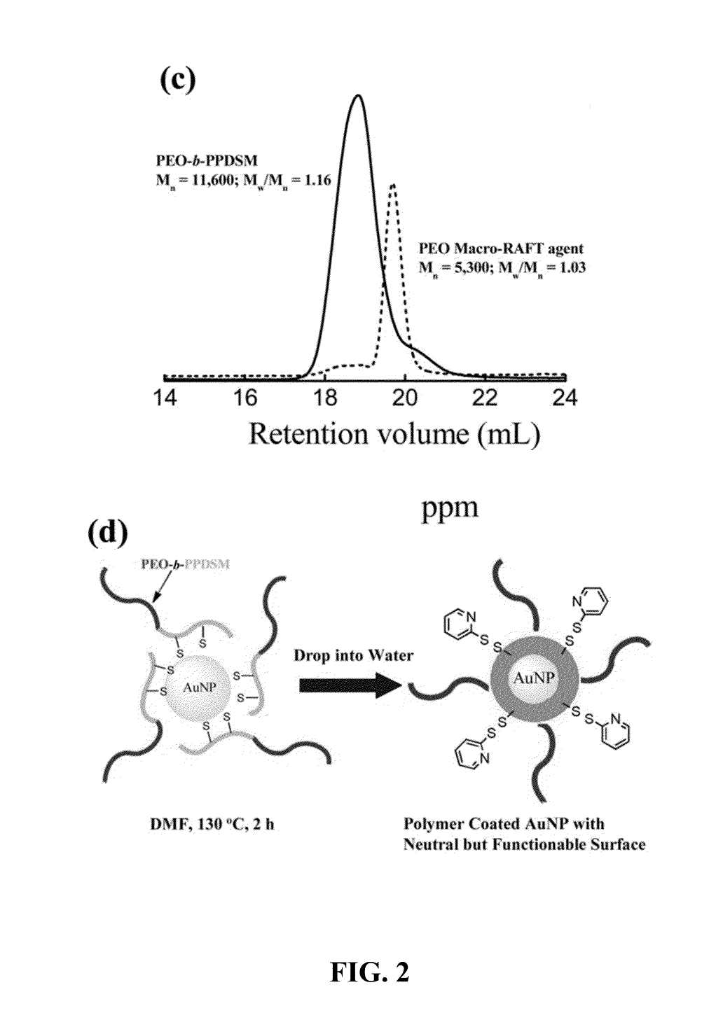

The thiol-reactive amphiphilic block copolymer poly(ethylene oxide)-block-poly(pyridyldisulfide ethylmethacrylate) (PEO-b-PPDSM) contains pyridyldisulfide functional groups, as shown in the scheme of FIG. 2a, was synthesized by reversible addition fragmentation chain transfer (RAFT) polymerization using PEO (Mₙ 5000 g/mol) macro-RAFT agent.

All the reagents used for synthesis of thiol-reactive amphiphilic block copolymer polyethylene oxide)-block-poly(pyridyldisulfide ethylmethacrylate) (PEO-b-PPDSM)were purchased from Aldrich chemical company and were used as received, unless otherwise mentioned. ¹H and ¹³C NMR were taken in Varian 400 MHz NMR spectrometer, UV visible spectra were recorded in a BioTek micro plate reader (Synergy 2) for aqueous solutions and UV-3600 (Shimadzu) for organic solutions. Molecular weight and molecular weight distribution of the copolymer was estimated by gel permeation chromatography (GPC) with THF as the eluent (flow rate=1.0 mL/min) using PS standard and UV detector. A series of three linear Styragel columns: HR0.5, HR1, and HR4 and a column temperature of 40° C. were used. The nanoparticles hydrodynamic size and zeta potential were measured using a dynamic light scattering (DLS) instrument (Malvern Zeta Sizer Nano S-90) equipped with a 22 mW He—Ne laser operating at γ=632.8 nm. The gold nanoparticles were viewed by transmission electron microscopy (TEM) (Philips CM-100 60 kV). The polymer coating was viewed through negative staining with OsO₄. Monomer PDSM was synthesized following previously reported procedure (Biomacromolecules, (2008) 9, 1934). PEO macro-RAFT agent was synthesized following literature reported procedure (Macromolecules, (2001) 34, 2248).

Synthesis of HydroxyethylpyridYl Disulfide (Compound 1):

Aldrithiol-2, (15 g, 0.068 mol) was dissolved in 75 mL of methanol. 1 mL of glacial acetic acid was then added. To this mixture, a solution of mercaptoethanol (2.65 g, 0.034 mol) in 25 mL methanol was added drop-wise at room temperature in 0.5 h under continuous stirring. Once the addition was over, the reaction mixture was stirred at room temperature overnight. The stirring was stopped and the solvent was evaporated to get the crude product as yellow oil. The crude product was then purified by column chromatography using silica gel as stationary phase (silica gel 60 A, 230-400 mesh) and mixture of ethyl acetate/hexane as eluent. The purification was monitored by TLC. The excess aldrithiol came out first at 15% ethyl acetate/hexane mixture, then the polarity of the eluent was increased to 40% ethyl acetate/hexane to get the desired product as pale yellow oil. Yield: 77%. ¹H NMR: (CDCl₃, 400 MHz), δ (ppm): 8.50 (m, 1H, aromatic proton ortho-N), 7.59 (m, 1H, aromatic proton meta-N), 7.42 (m, 1H, aromatic proton para-N), 7.15 (m, 1H, aromatic proton, ortho-disulfide linkage), 5.61 (b, 1H, HOCH2CH2—S—S), 3.80 (t, 2H, —S—S—CH2CH2OH), 2.95 (t, 2H, —S—S—CH2CH2OH).

Synthesis of Pyridyldisulfide Ethymethacrylate (PDSM):

To a solution of compound 1 (4.88 g, 26.0 mmol) in 20 mL of dry dichloromethane was added 3.95 g (39.0 mmol) of triethylamine and the mixture was cooled in an ice-bath. To this cold mixture, a solution of methacryloyl chloride (4.08 g, 39.0 mmol) in 10 mL of dry dichloromethane was added drop-wise with continuous stirring. After the addition was over in about 0.5 hour, the mixture was stirred at room temperature for 6 hours in an ice bath. The stirring was stopped and the solid was removed by filtration. The filtrate was washed with 3×30 mL distilled water and then 30 mL brine. The organic layer was collected, dried over anhydrous MgSO₄ and concentrated by rotary evaporation at room temperature to get the crude product as pale yellow oil. It was then purified by column chromatography using silica gel as stationary phase and mixture of ethyl acetate/hexane as eluent. The purification was monitored by TLC. The pure product was collected at 25% ethyl acetate/hexane. Yield: 82%. ¹H NMR: (CDCl₃, 400 MHz), δ (ppm): 8.44 (m, 1H, aromatic proton ortho-N), 7.67 (m, 2H, aromatic proton meta-N and para-N), 7.09 (m, 1H, aromatic proton, orthodisulfide linkage), 6.01 (d, 1H, vinylic proton, cis-ester), 5.56 (d, 1H, vinylic proton, trans-ester) 4.38 (t, 2H, —S—S—CH2CH2O—), 3.08 (t, 2H, —S—S—CH2CH2O—), 1.92 (s, 3H, methyl proton of the methacryloyl group).

Synthesis of Dithiobenzoic Acid (DTBA):

To a thoroughly dried 500 mL, three-necked round-bottomed flask equipped with a magnetic stir bar, addition funnel (250.0 mL), thermometer, and rubber septum for liquid transfers was added sodium methoxide (25% solution in methanol, 108 g, 0.5 mol) Anhydrous methanol (125 g) was added to the flask, followed by rapid addition of elememtal sulfur (16.0 g, 0.5 mol). Benzyl chloride (31.5 g, 0.25 mol) was then added dropwise via the addition funnel over a period of 1 hour, at room temperature under a dry nitrogen atmosphere. The reaction mixture was heated to reflux in an oil bath for 10 h. After this time, the reaction mixture was cooled to 7° C. using an ice bath. The precipitated salt was removed by filtration and the solvent removed in vacuo. To the residue was added deionized water (250 mL). The solution was then transferred to a 2 L reparatory funnel. The crude sodium dithiobenzoate solution was washed with diethyl ether (3—100 mL). Diethyl ether (100 mL) and 1.0 N HCl (250 mL) were added, and dithiobenzoic acid was extracted into the ethereal layer. Deionized water (250 mL) and 1.0 N NaOH (300 mL) were added, and sodium dithiobenzoate was extracted to the aqueous layer. This washing process was repeated one more time to finally yield a solution of sodium dithiobenzoate.

Synthesis of Di(thiobenzoyl) Disulfide:

Potassium ferricyanide (III) (32.93 g, 0.1 mol) was dissolved in deionized water (500.0 mL). Sodium dithiobenzoate solution (350 mL) was transferred to a 1 L conical flask equipped with a magnetic stir bar. Potassium ferricyanide solution was added dropwise to the sodium dithiobenzoate via an addition funnel over a period of 1 h under vigorous stirring. The red precipitate was filtered and washed with deionized water until the washings became colorless. The solid was dried in vacuo at room temperature overnight.

Synthesis of 4-Cyanopentanoic Acid Dithiobenzoate (CPAD):

To a 250 mL round-bottomed flask was added anhydrous ethyl acetate (80.0 mL). To the flask was added dry 4,4-azobis(4-cyanopentanoic acid) (5.84 g, 21.0 mmol) and di(thiobenzoyl) disulfide (4.25 g, 14.0 mmol). The reaction solution was heated at reflux for 18 h. The ethyl acetate was removed in vacuo. The crude product was isolated by column chromatography using ethyl acetate/hexane (2/3) as eluent. Fractions with only one band monitored by TLC that were red in color were combined and dried over anhydrous sodium sulfate overnight. The solvent mixture was removed in vacuo, whereupon it crystallized. The target compound was recrystallized from benzene. Yield: 66%. ¹H NMR: (CDCl₃, 400 MHz), δ (ppm): 7.4-8.0 (aromatic protons labeled with 1, 2, and 3), 2.5-3.0 (methylene protons labeled with 4, and 5), 2.0 (methyl protons labeled with 6). ¹³C NMR (Figure S4): (CDCl₃, 400 MHz) was further confirmed the structure as the peaks are assigned and labeled in the spectrum.

Synthesis of PEO Macro-RAFT Agent:

In a 250 mL one-neck round-bottom flask equipped with a magnetic stirring bar, PEO-OH (10.0 g) was dissolved in 150 mL of toluene. After azeotropic distillation of 10 mL of toluene at reduced pressure to remove traces of water, 0.5735 g of CPAD and 0.0643 g of 4-dimethylaminopridine (DMAP) were added. When the solution was homogenized by stirring, 1.1600 g of 1,3-dicyclohexylcarbodiimide (DCC) was added in portions. The reaction mixture was stirred at room temperature for 3 days. The precipitated urea was filtered. PEO-based macro-RAFT agent with pink color was obtained by precipitation of the filtrate into excess of diethyl ether three times, and then dried under vacuum at room temperature for 2 days. Yield: 93%. ¹H NMR: (CDCl₃, 400 MHz), δ (ppm): 7.3-7.9 (aromatic protons), 4.2 (methylene protons of newly formed ester groups), 3.42-3.63 (methylene protons of PEG repeat units), 2.32-2.55 (methylene protons of CPAD), 1.9 (methyl protons of CPAD).

Synthesis of Block Copolymer PEO-b-PPDSM:

The polymerization was performed in a schlenk flask with a magnetic stirring bar. The polymerization procedure is as follows. PDSM (1.03 g, 4 mmol), PEO-CTA (0.80 g, 0.16 mmol), and AIBN (6.3 mg, 0.04 mmol) were dissolved in DMAc (10 mL). The homogenized reaction mixture was subjected to four freeze-pump-thaw cycles to remove oxygen. The flask was then immersed into an oil bath preheated to 70° C. to start the polymerization. After 12 h, the reaction flask was quenched into the mixture of dry ice/2-propanol to stop the polymerization. After thawing, the solution was precipitated three times in diethyl ether and then dried in vacuo.

The block copolymer structure was confirmed by the ¹H NMR spectrum as shown in FIG. 2b. The spectrum showed the characteristic peaks from both PEO block (peak a) and PDSM block (peaks b, c, d, e, and f). The proton number of each peak showed on the spectrum for PDSM block matches well with the expected structure, revealing the absence of any significant transfer reaction to the pyridyldisulfide containing side groups (Biomacromolecules 2008, 9, 1934). It is estimated that the block copolymer contains ˜20 PDSM units based on the integration of peak f and peak a. The block copolymer structure was also confirmed by gel permeation chromatography (GPC) with expected elution peak shifted toward to the higher molecular weight in the elution profile (Mₙ 11,600 g/mol) and the low polydispersity index (PDI, 1.16) as shown in FIG. 2c. While the present invention is not limited to any particular mechanism, and an understanding of the mechanism is not necessary to practice the invention, it is believed that one of the unique characteristics of this copolymer is that it contains functional groups of multiple disulfide bonds on PDSM block which could interact with gold nanoparticles through multiple Au—S binding sites to result in stable and single dispersed gold nanoparticles in aqueous solution as shown in FIG. 2d.

Encapsulation of Gold Nanoparticles Using PEO-b-PPDSM:

In this Example, colloidal suspension of gold nanoparticles was used in acetone made by femtosecond laser ablation. After a couple of days aging, the top clear red solution was transferred and mixed with 2 mL of dimethylformamide (DMF). Acetone was evaporated under reduced pressure to form a concentrate gold solution in DMF. One mL of gold solution (20 μM in DMF) was mixed with 1 mL of PEO-b-PPDSM solution (50 mg/mL in DMF) in a 15 mL flask equipped with a magnetic stirring bar with gentle stirring at room temperature for more than 8 hours. Then the temperature was increased to corresponding temperatures in an oil bath for pre-set time points (typically 2 hours). After cooling to room temperature slowly, the resultant mixture was added dropwise to 20 mL of deionized water under magnetic stirring. The block copolymer encapsulated gold nanoparticles were isolated through three times centrifugation using an Eppendorf 5424 centrifuge at 15,000 rpm for 30 minutes. Supernatant was removed by careful pipetting, and the AuNP was resuspended in deionized water. Also, the formed amphiphilic block polymer coated gold nanoparticles can be extracted from the solution and exist in the form of a powder

Various chemical functional groups, such as thiol, amine, disulfide, and phosphine, possess a high affinity for the surface of gold nanoparticles. Thiol groups are considered to show the highest affinity for gold surfaces, approximately 200 kJ/mol, and therefore a majority of gold nanoparticle surface functionalization occurs through using ligand molecules having thiol groups which bind to surfaces of gold nanoparticles via a thiol-Au bond.

In addition to poly(ethylene oxide) (PEO) polymer, other polymers selected from but not limited to poly(2-(methacryloyloxy)ethyl phosphorylcholine), poly(2-(dimethylamino)ethyl methacrylate), poly(acrylic acid), and poly(ethylene glycol) could also be used as hydrophilic polymer block of amphiphilic block copolymer.

In addition to poly(pyridyldisulfide ethylmethacrylate) (PPDSM) polymer, other polymers selected from but not limited to poly(methyl methacrylate), polystyrene, poly(N-isopropylacrylamide), and poly(methacrylic acid) could also be used as hydrophobic polymer block of amphiphilic block copolymer.

This Example reveals that heat treatment of the gold nanoparticles and polymer mixture during preparation process provides three advantages. First, heat treatment results in uniform nanoparticle size by causing the smaller gold nanoparticles to grow to the same size as the larger ones. Second, heat treatment also increased coating efficiency with enhanced Au—S binding. Finally, heat treatment enabled single nanoparticle formation when transferring the mixture of the polymer and gold nanoparticles into aqueous solution. In contrast, variable particle size, low coating efficiency, and multiple gold nanoparticles inside polymer micelles were observed at room temperature.

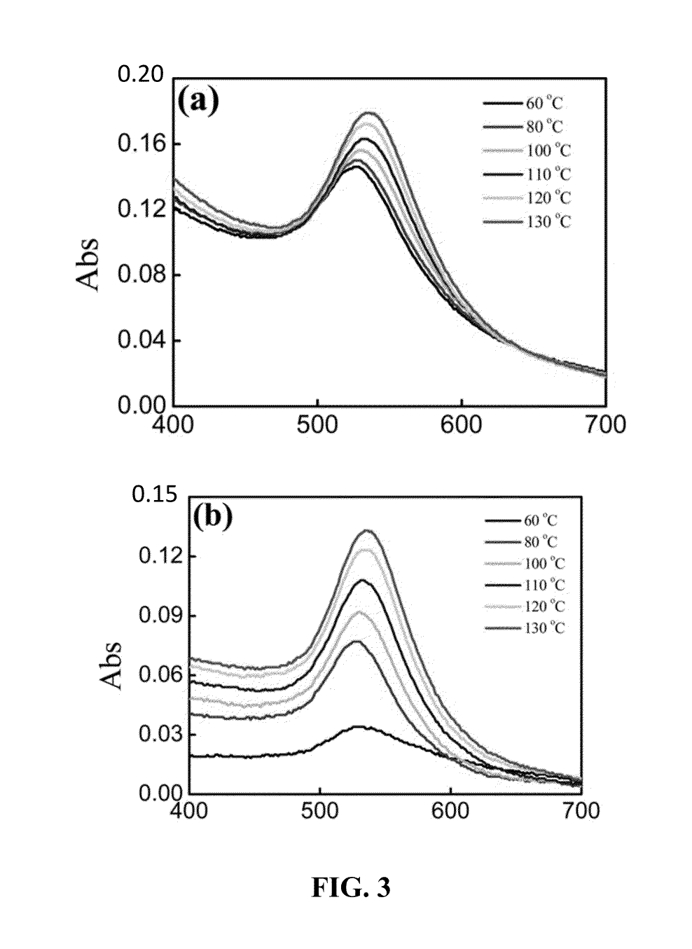

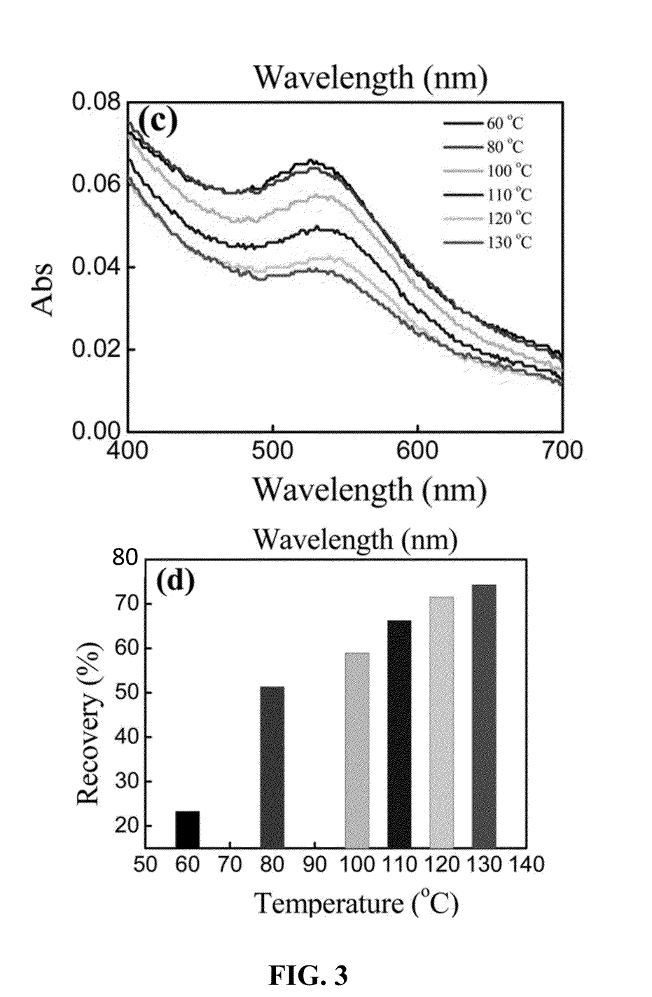

FIG. 3 shows the effect of heat treatment on the nanoparticles at different temperatures in the range from 60 degree to 130 degree. FIG. 3a shows the absorption spectrum after transferring the mixture to water before centrifugation. The results revealed that the absorption density peak from gold nanoparticles was consistently increased after heating at increased temperature in the range from 60 degree to 130 degree when the same concentration of gold polymer mixture was transferred into the same amount of water. According to Lambert-Beer law A=εbC, where A is the absorption intensity, ε is the extinction coefficient, b is the pass length, and C is the concentration of gold nanoparticles, the increasing absorption suggested the increase of extinction coefficient by the increase of the size of gold nanoparticles (Colloid Surface B 2007, 58, 3). This phenomenon was also revealed by the red shift of the absorption after heating at increased temperatures. The size growth of gold nanoparticles after heat treatment was further and more obviously confirmed by the absorption spectrum of purified gold nanoparticles after three times of centrifugation as shown in FIG. 3b. During centrifugation, only gold nanoparticles with sufficient size can be isolated. From FIG. 3b, one can conclude that the higher the treated temperature, the more gold nanoparticles are isolated through centrifugation. This is because more large gold nanoparticles were generated under higher temperature treatment through small gold nanoparticles growing into larger ones. In contrast, fewer of the smaller gold nanoparticles (<˜2 nm) grew larger under lower temperature treatment; thus they remained in supernatant and showed high absorption (FIG. 3c). The recovery percentage was defined by the ratio of absorption peak after centrifugation to absorption peak before centrifugation as shown in FIG. 3d. The data revealed that the recovery of gold nanoparticles was dramatically increased after heating with higher temperature with 75% recovery at 130° C. compared to ˜23% at 60° C. after heating for 2 hrs. These data suggest that heat treatment at higher temperature can increase coating efficiency through further growth of gold nanoparticles and enhancement of polymer gold nanoparticle interaction (Au—S) (Chemphyschem 2008, 9, 388).

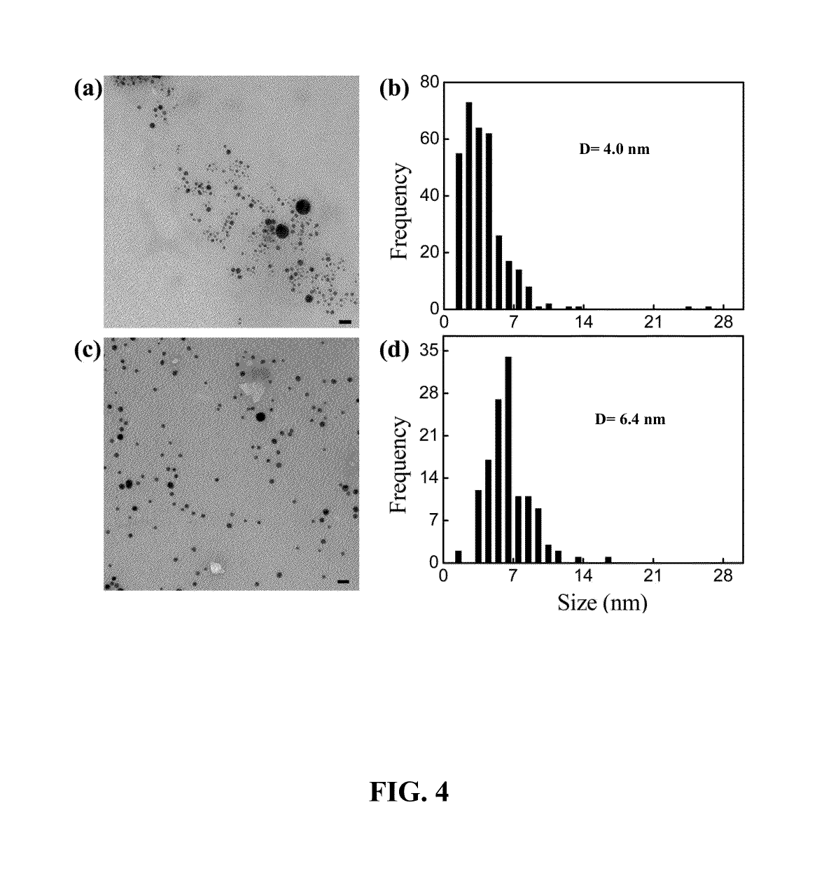

Transmission electron microscopy (TEM) was used to visualize the uniform sized single gold nanoparticles encapsulated with the copolymer during the heating process (typically at 130° C.). It was found that in addition to the increased size of these nanoparticles when heated at elevated temperature in the range from 60 degree to 130 degree, gold nanoparticles also become more uniform as smaller gold nanoparticles are enlarged. This could be seen by comparing TEM images before and after heat treatment (FIG. 4). Analysis of particles size from ImageJ shows the average gold size increased from 4.0 nm to 6.4 nm (average based on more than 100 gold nanoparticles), and no smaller nanoparticles remained. This result is consistent with optical spectrum study as discussed above. By comparing the two TEM images, it was also observed that single gold nanoparticles after heat treatment were separated from each other on TEM grid, implying that the amphiphilic block copolymer poly(ethylene oxide)-block-poly(pyridyldisulfide ethylmethacrylate) (PEO-b-PPDSM) already bound onto the gold nanoparticles during the heat process.

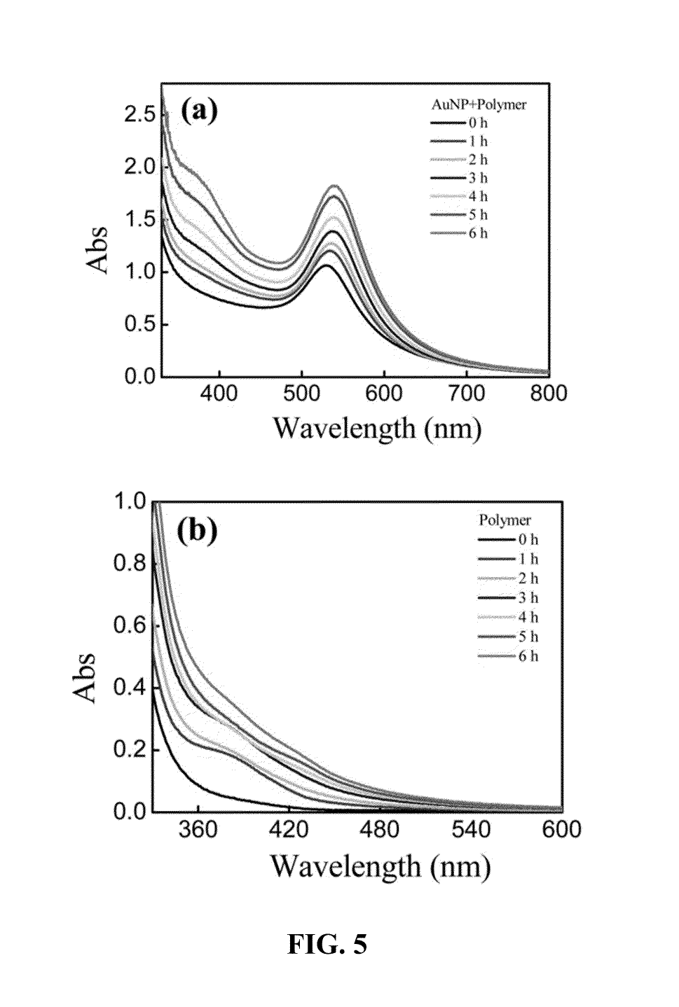

While the present invention is not limited to any particular mechanism and an understanding of the mechanism is not necessary to practice the invention, the Au—S enhanced binding is probably attributed to the exposure of thiol groups on polymer chains by reducing disulfide bonds, because the optical spectra revealed the release of pyridine-2-thione after heat treatment (FIG. 5). This heating process at higher temperature could potentially solve the limitation of the nanoparticles with wider size distribution made by laser ablation (J. Phys. Chem. C 2010, 114, 15931), since the bound polymer can mediate and control the further growth of gold nanoparticles.

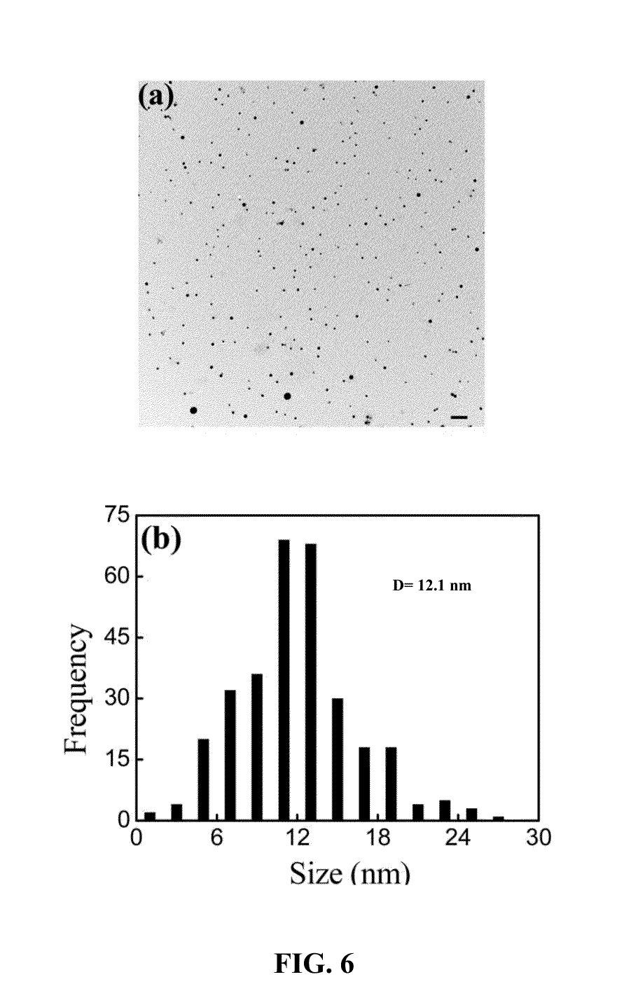

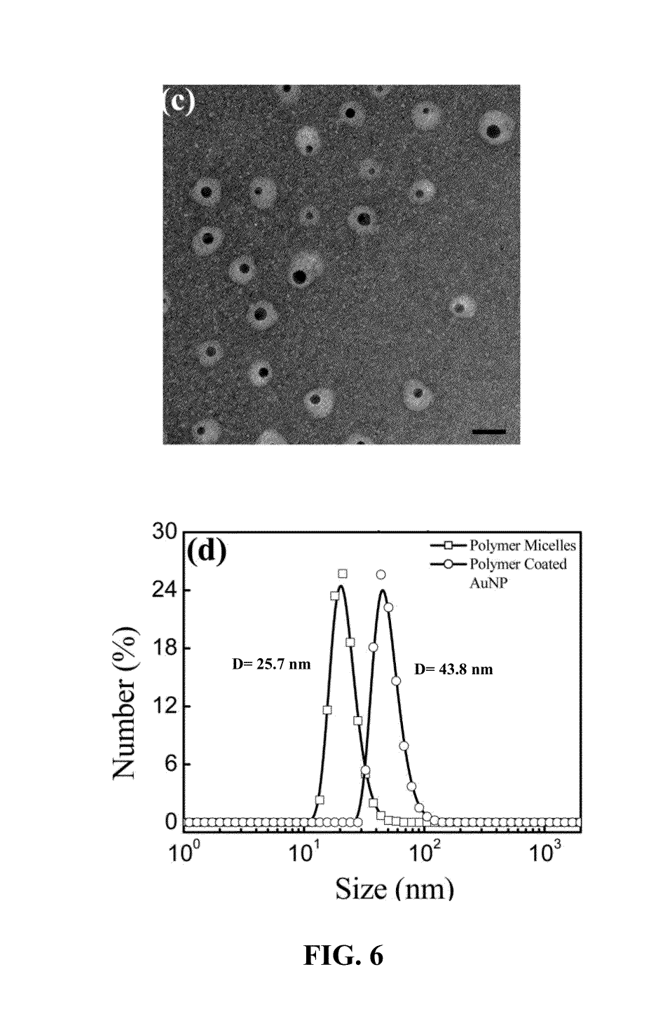

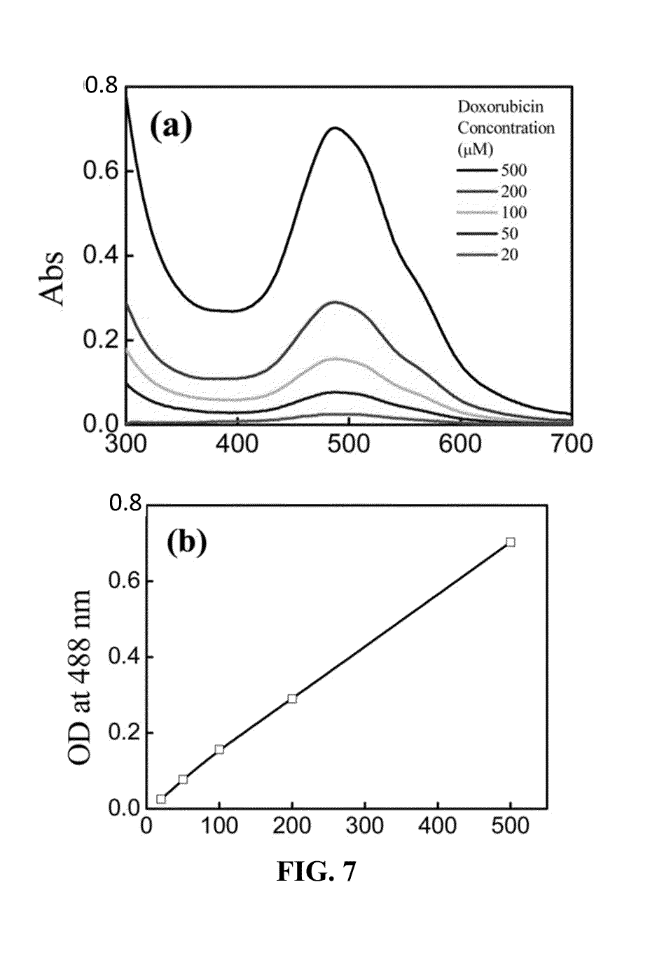

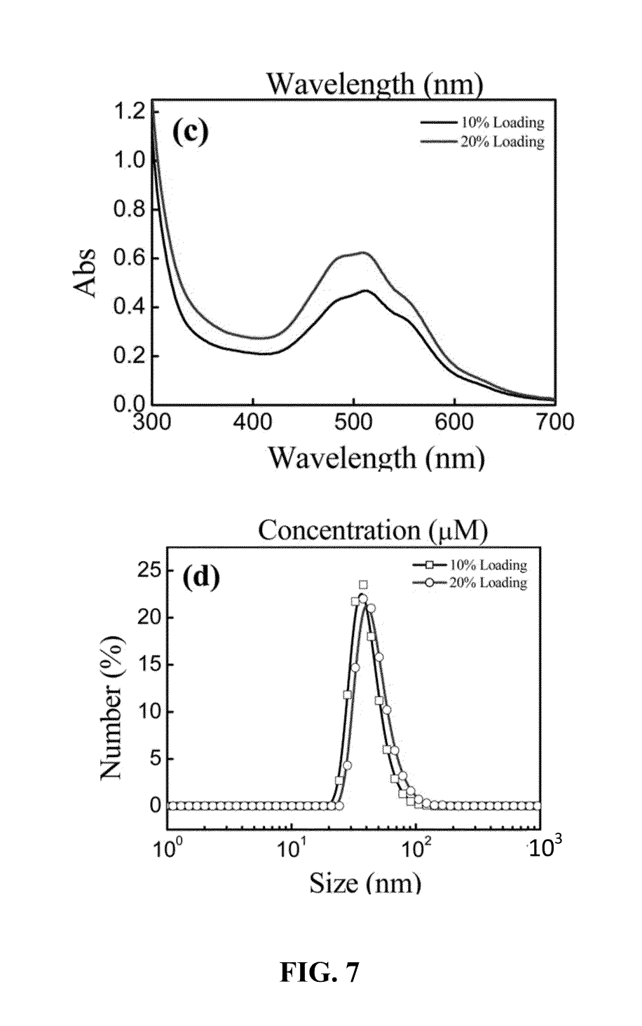

After transferring the mixture of gold nanoparticle and polymer into water followed by successful purification using centrifugation, the TEM image in FIG. 6a (no negative staining) showed that the gold nanoparticles are singly dispersed with an average core size at ˜12 nm as shown in FIG. 6b, which is larger than that before centrifugation (˜6.4 nm), implying the loss of some smaller particles during purification. The polymer coating around each gold nanoparticle was further revealed by the negative staining as shown in FIG. 6c. The polymer shell (˜8 nm thick) is composed of hydrophilic PEO out layer and collapsed hydrophobic PPDSM inner layer, which have the potential to encapsulate hydrophobic therapeutic drugs (Nano Lett. 2006, 6, 2427). This is true as the data confirmed that the composite nanoparticles have at least 20% of doxorubicin (neutral) loading efficiency (based on polymer mass) (FIG. 7).

FIG. 6d shows the average hydrodynamic size of both polymeric micelles only and polymer encapsulated gold nanoparticles measured by dynamic light scattering (DLS). Dynamic light scattering (DLS) is considered by many to be a standard method for measuring the average nanoparticle size because of its wide availability, simplicity of sample preparation and measurement, relevant size range measurement from 1 nm to about 2 um, speed of measurement, and in situ measurement capability for fluid-born nanoparticles. The data revealed that the hydrodynamic size of composite nanoparticles was increased from ˜26 (pure micelles) to 44 nm after encapsulation of gold core as shown in FIG. 3d, which is similar to the overall nanoparticle size revealed by negative staining The monodispersed amphiphilic polymer coated gold nanoparticles with smaller overall size (5-40 nm) are favorable for in vivo applications due to a longer mean blood circulation time and better tissue penetration (Angew. Chem. Int. Ed. 2008, 47, 5122).

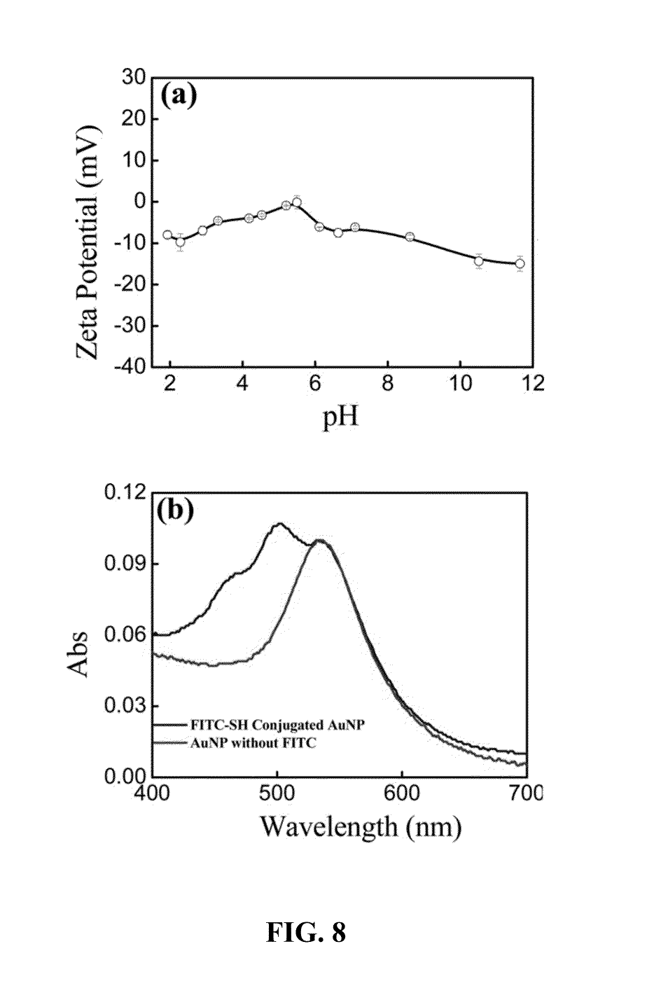

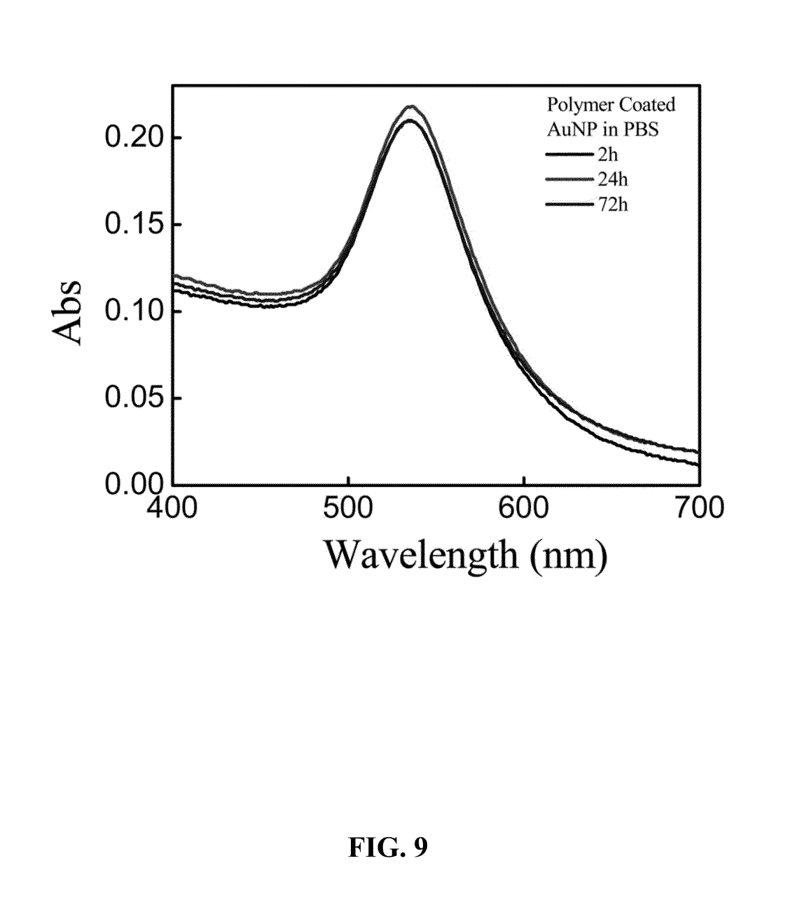

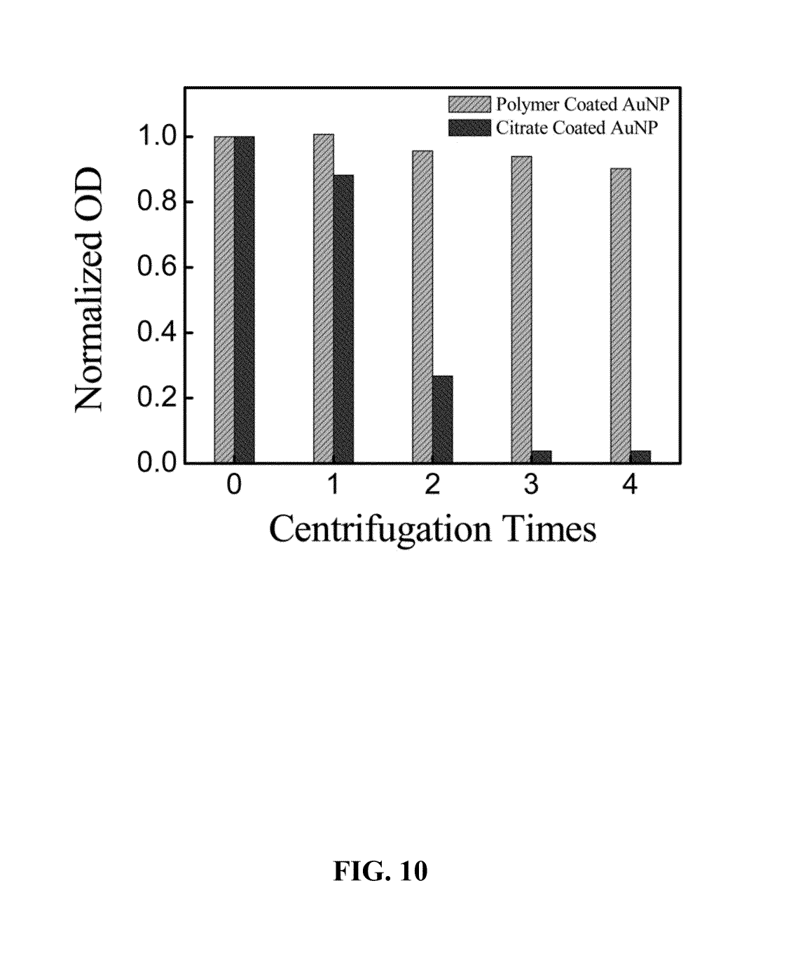

Since there are no charged groups with amphiphilic block copolymer poly(ethylene oxide)-block-poly(pyridyldisulfide ethylmethacrylate) (PEO-b-PPDSM), the coated gold nanoparticles are expected to have neutral surfaces. Zeta potential was applied to test this hypothesis as shown in FIG. 8a. The data showed that these polymer coated gold nanoparticles have slight negative zeta potential (−10-0 mV) at wide pH in the range from 2-12. This neutral property of nanoparticles has advantages to reduce nonspecific binding to tissues or other biological components in both in vitro and in vivo applications (Small 2010, 6, 12). Although the zeta potential is close to zero, the copolymer coated gold nanoparticles showed good stability in physiological conditions and various pH conditions, which is a prerequisite for in vivo applications. FIG. 9 shows the long term stability of polymer coated gold nanoparticles in PBS revealed by monitoring the absorption spectrum over three days without obvious decrease in absorption. Compared to the PEGylated gold nanoparticles stability in regular PBS which was only monitored for 20 minutes (P. Natl. Acad. Sci. USA 2010, 107, 1235), gold nanoparticles coated with PEO-b-PPDSM shows more promising stability to protect them from aggregation in vivo. In addition, the stability was also confirmed by more than 90% recovery after as least four centrifugation processes as shown in FIG. 10. This stability after repeated centrifugation will provide significant advantage for further modification compared to other gold nanoparticles with different coatings. In contrast, the citrate stabilized gold nanoparticles cannot tolerate two centrifugation-washing processes, as revealed by significant loss of absorption from gold nanoparticles due to aggregation (Soft Matter. 2011, 7, 3246). It is worth noting that the polymer layer around each gold nanoparticle coated with this amphiphilic block copolymer PEO-b-PPDSM contains multiple disulfide bonds and so very likely multiple Au—S interactions, which provide potential stability against possible dilution.

The amphiphilic block copolymer coated single dispersed gold nanoparticles are stable in phosphate buffered saline (PBS) buffer means a variation of less than plus or minus 10% of the localized surface plasmon resonance intensity of said amphiphilic block copolymer coated single dispersed gold nanoparticles in phosphate buffered saline (PBS) buffer after being in phosphate buffered saline (PBS) buffer for 72 hours at 25° C., compared to a localized surface plasmon resonance intensity of said amphiphilic block copolymer coated single dispersed gold nanoparticles measured immediately after preparation of said amphiphilic block copolymer coated single dispersed gold nanoparticles in phosphate buffered saline (PBS) buffer; and a variation of less than 4 nanometers shift of the wavelength of localized surface plasmon resonance of said amphiphilic block copolymer coated single dispersed gold nanoparticles in phosphate buffered saline (PBS) buffer after being in phosphate buffered saline (PBS) buffer for 72 hours at 25° C., compared to a wavelength of localized surface plasmon resonance of said amphiphilic block copolymer coated single dispersed gold nanoparticles measured immediately after preparation of said amphiphilic block copolymer coated single dispersed gold nanoparticles in phosphate buffered saline (PBS) buffer.

### Example 2

One of the most important advantages of these polymer coated gold nanoparticles is that the resultant nanoparticles have neutral surfaces but can be further conjugated without any modifications to the nanoparticles. It was hypothesized that surface functionlization can be achieved through thiol-disulfide exchange reactions with the PDSM groups (J. Am. Chem. Soc. 2010, 132, 8246). To test this, polymer coated gold nanoparticles were treated with thiol-modifed FITC.

Preparation of Thiol-Modified FITC:

A mixture of FITC (20 mg, 0.052 mmol), cystamine dihydrochloride (6.0 mg, 0.026 mmol) and triethylamine (26.0 mg, 0.26 mmol) was dissolved in DMSO (800 μl) and stirred for 4 h. To this reaction mixture was added tris(2-carboxyehtyl)phosphine hydrochloride (17.6 mg, 0.062 mmol) and stirred for 1 hour. The resultant mixture was precipitated in ethyl ether and washed with water. The crude product was used for polymer coated gold nanoparticles surface modification without further purification.

Functionalization of Gold Nanoparticles Coated with PEO-b-PPDSM with FITC:

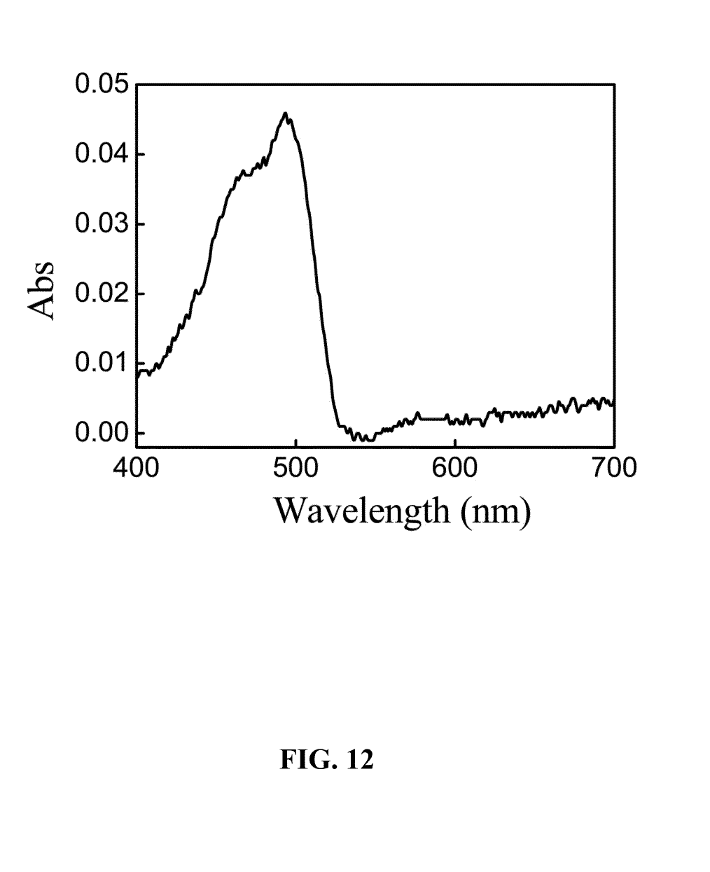

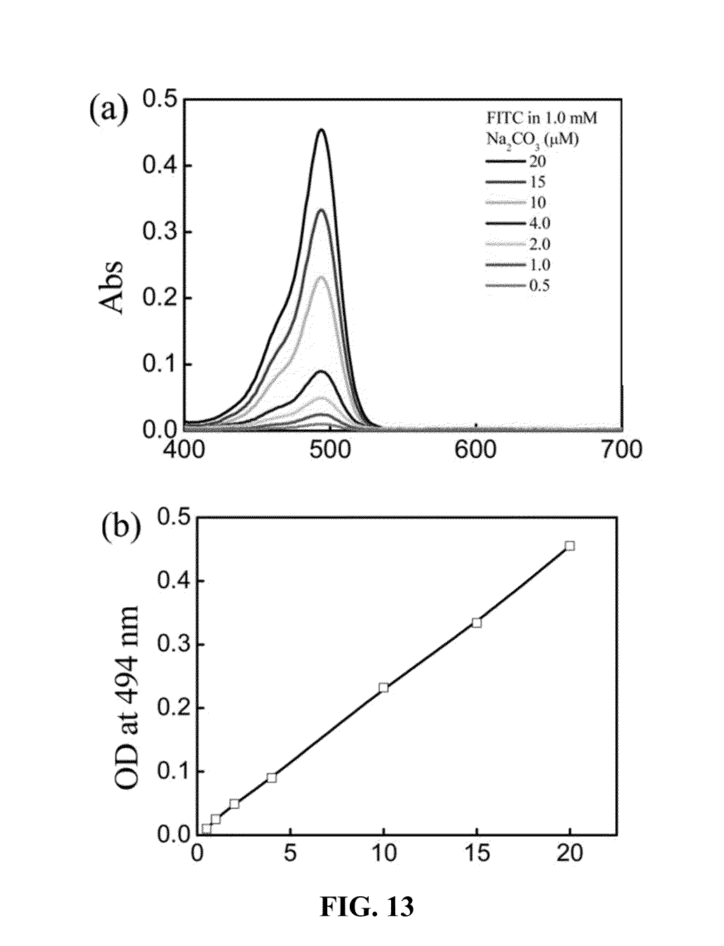

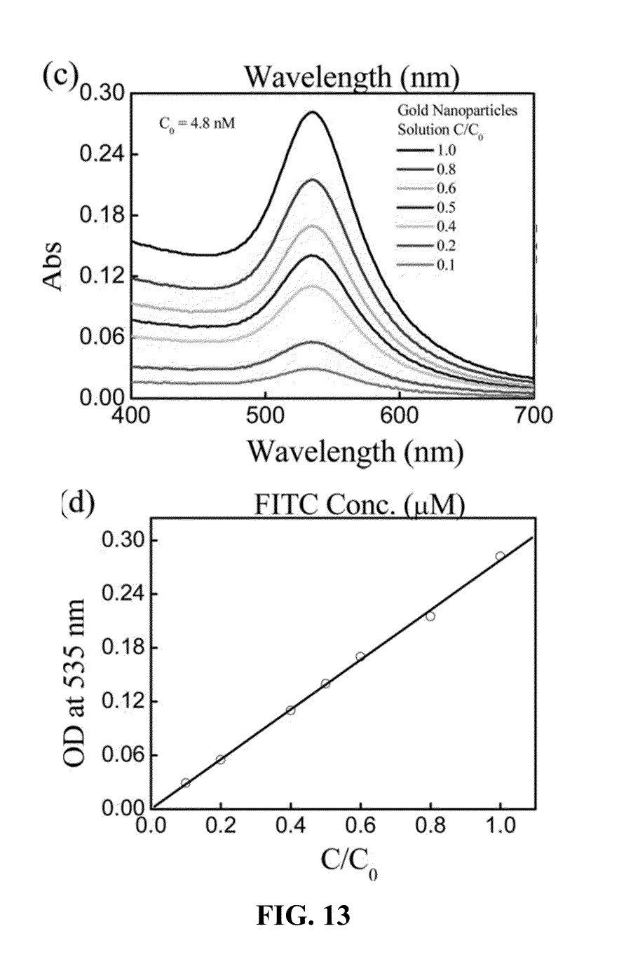

One mg of FITC or thiol-modified FITC was dissolved in 100 μL of DMF and then 1 mL of polymer coated gold nanoparticles (4.8 nM) in water was added. 0.1 M NaOH was used to adjust the pH until the solution is clear. The mixture solution was stirred overnight at room temperature. Non-conjugated dye molecules were removed by ultrafiltration and re-suspended using 1.0 mM sodium carbonate until there is no detectable dye in the filtrated solution (five times) using a nanosep® filter (Pall Corp.) with a molecular weight cutoff of 30,000 g mol⁻¹. The concentration of a gold nanoparticles solution without FITC modification was adjusted to match the same optical density at 535 nm as FITC modified one to show the FITC signal after subtraction as shown in FIG. 12. A calibration curve of gold nanoparticles and FITC in 1.0 mM sodium carbonate was created to estimate the number of FITC conjugated on each gold nanoparticles as shown in FIG. 13.

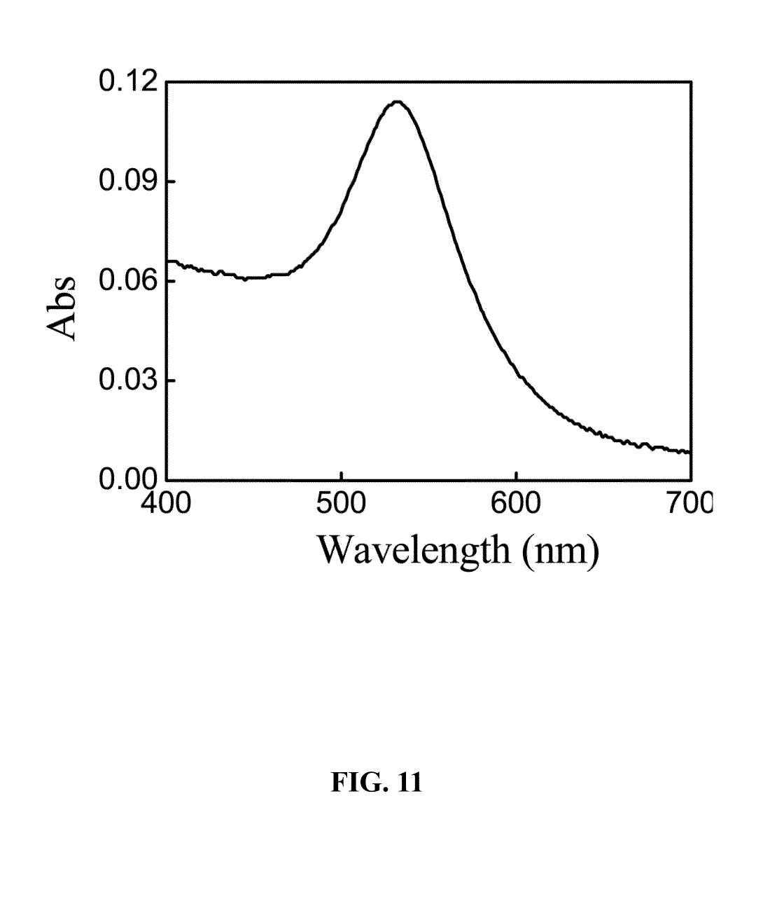

FIG. 8b shows the specific absorption peak from FITC at 494 nm which shows a different absorption level for gold nanoparticles, indicating the polymer coated gold nanoparticles were covalently functionalized with thiol-modified FITC by disulfide linkage. This is also confirmed by comparing the absorption spectrum of gold nanoparticles treated with FITC but without thiol modification, where a signal from FTIC is absent after purification (FIG. 11). After subtraction from absorption spectrum of unmodified gold nanoparticle solution, the conjugated FITC absorption spectrum was clearly seen (FIG. 12). It is estimated that ˜1200 FITC molecules were conjugated on each polymer coated gold nanoparticle based on the calibration curves of both FITC and gold nanoparticles in aqueous solution (FIG. 13).

Thus, while only certain embodiments have been specifically described herein, it will be apparent that numerous modifications may be made thereto without departing from the spirit and scope of the invention. Further, acronyms are used merely to enhance the readability of the specification and claims. It should be noted that these acronyms are not intended to lessen the generality of the terms used and they should not be construed to restrict the scope of the claims to the embodiments described therein. Additionally, all references cited herein are incorporated by reference.

We claim:

1. A method of producing stable amphiphilic block copolymer coated single dispersed nanoparticles comprising:
a) mixing a solution of amphiphilic block copolymer with a colloidal suspension of nanoparticles to generate a mixture, wherein said amphiphilic block copolymer comprises at least one functional group having an affinity for said nanoparticles;
b) treating said mixture at a temperature of above about 60 degrees Celsius to generate a treated mixture; and
c) adding at least a portion of said treated mixture to deionized water such that a solution is generated that comprises amphiphilic block copolymer single dispersed nanoparticles.

2. The method of claim 1, wherein said nanoparticles comprise gold nanoparticles.

3. The method of claim 1, further comprising d) removing said amphiphilic block copolymer coated single dispersed nanoparticles from said solution and mixing with deionized water.

4. The method of claim 1, wherein said treated mixture is added dropwise to said deionized water.

5. The method of claim 1, wherein said deionized water is in circular motion when said treated mixture is added thereto.

6. The method of claim 1, wherein said temperature is above 100 degrees Celsius.

7. The method of claim 1, wherein said temperature is about 60-160 degrees Celsius.

8. The method of claim 1, wherein said mixing in step a) is conducted at about room temperature.

9. The method of claim 1, wherein said treated mixture is cooled to about room temperature prior to step c).

10. The method of claim 1, wherein said amphiphilic block copolymer comprises a polymer selected from the group consisting of: poly(2-(methacryloyloxy)ethyl phosphorylcholine), poly(2-(dimethylamino)ethyl methacrylate), poly(acrylic acid), poly(ethylene oxide), poly(ethylene glycol),poly(methyl methacrylate), polystyrene, poly(pyridyldisulfide ethylmethacrylate), poly(N-isopropylacrylamide), and poly(methacrylic acid).

11. The method of claim 1, wherein said amphiphilic block copolymers comprise hydrophilic or hydrophobic polymer block having degree of polymerization in the range from 1 unit to 100 units.

12. The method of claim 1, further comprising, prior to step a) preparing said colloidal suspension of nanoparticles by a top-down nanofabrication method using bulk metal as a source material.

13. The method of claim 12, wherein said top-down nanofabrication method comprises applying a physical energy source to said bulk metal, said physical energy source comprising at least one of mechanical energy, heat energy, electric field arc discharge energy, magnetic field energy, ion beam energy, electron beam energy, or laser beam energy.

14. The method of claim 1, wherein said colloidal suspension of nanoparticles comprises a population of nanoparticles wherein said nanoparticles have at least one dimension in the range of from 1 to 200 nanometers.

15. The method of claim 1, wherein said functional group comprises a thiol group, an amine group, a phosphine group, a disulfide group or a mixture thereof.

16. A composition comprising at least a portion of said amphiphilic block copolymer single dispersed nanoparticles prepared by the method of claim 1.

17. Amphiphilic block copolymer coated single dispersed nanoparticles which are stable in buffer solution comprising: a population of single nanoparticles encapsulated in a shell formed by said amphiphilic block copolymers, said amphiphilic block copolymers contains at least one functional group having an affinity for the surface of said nanoparticles in its hydrophobic part and wherein said amphiphilic block copolymers coated nanoparticles have electrically neutralized surfaces and provide capability for further functionalization via thiol-disulfide exchange reactions.

18. The amphiphilic block copolymer coated single dispersed nanoparticles of claim 17, wherein said functional group comprises a thiol group, an amine group, a phosphine group, a disulfide group or a mixture thereof.

19. The amphiphilic block copolymer coated single dispersed nanoparticles of claim 17, wherein said amphiphilic block copolymer comprises hydrophobic or hydrophilic polymer block having degree of polymerization in the range from 1 unit to 100 units.

20. The amphiphilic block copolymer coated single dispersed nanoparticles of claim 17, wherein said hydrophilic or hydrophobic polymer block of said amphiphilic block copolymer comprise a plurality of polymers selected from the group consisting of: poly(2-(methacryloyloxy)ethyl phosphorylcholine), poly(2-(dimethylamino)ethyl methacrylate), poly(acrylic acid), poly(ethylene oxide), poly(ethylene glycol), poly(methyl methacrylate), polystyrene, poly(pyridyldisulfide ethylmethacrylate), poly(N-isopropylacrylamide), and poly(methacrylic acid).

21. The amphiphilic block copolymer coated single dispersed nanoparticles of claim 17, wherein said nanoparticles have at least one dimension in the range of from 1 to 200 nanometers.

22. The amphiphilic block copolymer coated single dispersed nanoparticles of claim 17, wherein said amphiphilic block copolymer coated single dispersed nanoparticles are in powder form.

23. The amphiphilic block copolymer coated single dispersed nanoparticles of claim 17, wherein said nanoparticles comprise gold.
